(19) United States
(12) Patent Application Publication
(10) Pub. No.: US 2021/0299272 A1
(43) Pub. Date: Sep. 30, 2021

(54) METHODS AND COMPOSITIONS FOR DRUG DELIVERY

(71) Applicant: The Trustees of the University of Pennsylvania, Philadelphia, PA (US)

(72) Inventors: Vladimir Muzykantov, Bryn Athyn, PA (US); Jacob S. Brenner, Princeton Junction, NJ (US); Carlos H. Villa, Philadelphia, PA (US); Laura Ferguson, Philadelphia, PA (US); Elizabeth Hood, Philadelphia, PA (US)

(21) Appl. No.: 17/219,295

(22) Filed: Mar. 31, 2021

Related U.S. Application Data
(60) Provisional application No. 63/002,996, filed on Mar. 31, 2020.

(51) Int. Cl. A61K 47/69; A61K 47/10; A61K 45/06; C07K 16/28

(52) U.S. Cl. CPC A61K 47/6929; C07K 16/2896; A61K 45/06; A61K 47/10

(57) ABSTRACT

Compositions comprising a dual-targeted nanoparticle having a first targeting moiety and a second targeting moiety, wherein said first targeting moiety is a red blood cell (RBC)-targeting moiety are provided. In certain embodiment, the nanoparticles are bound to RBCs ex vivo. Also provided are methods of delivering selected drugs to target organs using these compositions for treatment of disease or for diagnostic imaging.

### STATEMENT REGARDING FEDERALLY SPONSORED RESEARCH OR DEVELOPMENT

This invention was made with government support under grant numbers HL143806 and HL138269 awarded by the National Institutes of Health. The government has certain rights in this invention.

### BACKGROUND OF THE INVENTION

A critical reason that drugs fail to gain regulatory approval is that off-target side effects limit dosing necessary to achieve therapeutic effect. A long-studied, key strategy to overcome this challenge has been the loading of drugs into nanometer-scale carriers (nanocarriers or nanoparticles) conjugated with ligands (affinity moieties) that specifically direct the nanocarrier to receptors in the target tissue. A broad array of ligand-guided nanocarriers have been developed, beginning with monoclonal antibody-conjugated liposomes (immunoliposomes) over 40 years ago.¹ Later iterations of ligand-based targeting have utilized antibody fragments, peptides, nucleic acids, and polysaccharides.^{2,3} Target organ uptake for these ligand-guided nanocarriers markedly exceeds that of untargeted ones, in some cases by orders of magnitude, showing high specificity of targeting.

Despite the improvement in drug localization by these ligand-guided nanocarriers, in nearly every case the majority of the initial dose is still delivered to off-target locations or cleared from circulation. The efficacy of delivery of ligand-guided nanocarriers in the target site usually does not exceed 5% percent of the initial injected dose (% ID) with an absolute “efficacy ceiling” achieved in animal studies peaking at 15-25% ID.⁴ A majority of nanocarriers end up outside the target tissue, even in the lungs, which have natural targeting advantages. The lung's capillary network provides a unique test bed for intravenously (IV)-delivered, ligand-targeted nanocarriers, due both to its enormous surface area and that it receives 100% of cardiac output after IV drug injection. Despite this ideal test bed and targeting highly-expressed endothelial cell adhesion molecules (CAMs), approximately 80% of the initial dose is cleared or retained in organs outside the lung.⁵⁻⁸ When the target site is reduced to a sub-organ level, as with a tumor, the delivery efficiency falls even further, with meta-analysis finding ˜0.7% ID reside in tumors and only minimal improvement using antibody targeting.⁴ Thus, ligand-conjugated nanocarriers show increased organ targeting compared to free drugs, but the target organ still receives only a minority fraction of the injected dose.

A continuing need in the art exists for new and effective compositions and methods for drug delivery using nanocarriers.

### SUMMARY OF THE INVENTION

Provided herein in one aspect is a composition comprising dual-targeted nanoparticles having a first targeting moiety and a second targeting moiety, wherein the first targeting moiety is a red blood cell (RBC)-targeting moiety. In certain embodiments, the composition further comprises RBCs bound to the nanoparticles ex vivo via said first targeting moiety. In certain embodiments, the nanoparticles are liposomes, nanogels, or polymeric nanoparticles. In certain embodiments, the first and/or second targeting moiety is an antibody or antibody fragment, carbohydrate, carbohydrate-binding compound, peptide, nucleic acid, or aptamer. In a particular embodiment, the first targeting moiety is an antibody specific for a glycophorin, optionally glycophorin A or Band 3, or an Rh antigen.

In certain embodiments, the nanoparticle composition provided herein includes a second targeting moiety that is specific for vascular endothelial cells, intravascular leukocytes, cells of reticuloendothelial system, an immune cell, cells and tissues accessible to RBC under pathological conditions such as hemorrhage and thrombosis, or an infectious microorganism. In certain embodiments, the second targeting moiety is specific for ICAM-1, PECAM-1, VCAM-1, transferrin receptor, or ACE.

In certain embodiments, the compositions provided are characterized by having at least one of (a) about 50 to about 200 bound nanoparticles per RBC; (b) about 5 to about 350 of said first targeting moiety per nanoparticle; and (c) about 2.5% to about 25% of targeting moieties on the nanoparticle surface comprise said first targeting moiety; (d) a total number of first and second targeting moieties per nanoparticle in the range or about 5 to 350; and (e) a particle size diameter of about 10 nm to about 1,000 nm. In certain embodiments, the nanoparticles are loaded with a drug.

Also provided herein are pharmaceutical compositions that include an aqueous suspension comprising dual-targeted nanoparticles.

In yet another embodiment, provided herein is a method for delivering a drug to a mammalian subject having a disease, the method comprising administering to a subject in need thereof a composition or pharmaceutical composition comprising dual-targeted nanoparticles. In certain embodiments, (a) the disease is ARDS, pulmonary arterial hypertension, pneumonia, interstitial lung disease, idiopathic pulmonary fibrosis, post-pulmonary embolism, pulmonary capilliaritis syndrome, stroke, emphysema, lung edema, or a viral or microbial infection; (b) the disease is ARDS and wherein the drug is one of more of albuterol, dexamethasone, and palifermin; and/or (c) the disease involves a selected mammalian organ, and the nanoparticle composition is administered intra-arterially or intravenously. In certain embodiments, the drug is delivered to one or more target organs, including but not limited to the lungs, brain, or heart. In certain embodiments, the dual-targeted nanoparticles are administered intravenously or intraarterially.

In yet a further embodiment, the composition comprising the dual-targeted nanoparticles is administered to a patient and circulating RBCs in the bloodstream of the patient bind to the nanoparticles via the first targeting moiety.

In yet a further embodiment, the method provided includes contacting the dual-targeted nanoparticles with RBCs ex vivo to bind RBCs to the nanoparticles via the first targeting moiety prior to administration to the subject, wherein the RBCs are present in a sample obtained from the patient or an autologous blood donor. In certain embodiments, the method includes separating or enriching RBCs present in the sample prior to binding to the dual-targeted nanoparticles.

Still other aspects and advantages of these compositions and methods are described further in the following detailed description of the preferred embodiments thereof.

### BRIEF DESCRIPTION OF THE DRAWINGS

FIG. 1A-FIG. 1F show characterization of dual-targeted vs endothelial and RBC targeted liposomes and their RBC binding capacity and effects. (FIG. 1A) DTRH liposome particle component scheme generalized, including targeting ligands and radioactive tracers; not to scale. (FIG. 1B) Schematic of DT RBC loading (left), the RBC-liposome complexes circulate through capillaries with close contact to CAMs (center), and (right) proposed transfer of DT liposomes bound to RBCs to an endothelial cell via CAM on EC cell surface. (FIG. 1C) Summary in vivo lung localization comparison by % injected dose (% ID) data of directly injected liposomes (L) and RH liposomes targeted to only RBC (RT), endothelial cells (ET) and by dual targeting (DT), compared to untargeted injected free drug localized in mice to lungs at 30 mins post injection. Enhancement of lung targeting by RH and further by DTRH, first by ⅓ as endothelial cell targeted (ET) liposome were adsorbed to RBCs (ET-RH) and further by 2-fold to 65% ID with the inclusion of RBC antibodies to RH equivalent liposomes. Error bars are standard deviation, n>=4 for all samples. (FIG. 1D) Liposome size and antibody coating characteristics. Sample naming conventions defined by coating ratios RBC:EC antibodies on liposomes: untargeted=non-antibody coated; ET=particles coated with EC targeted antibodies only, DT=dual-targeted with varied ratios, and RT=only RBC Ab. Size by hydrodynamic radius, indicates ET, DT, and RT formulations vary little by mean size with low polydispersity indices. (FIG. 1E and FIG. 1F) Verification of the extent and precision of DT antibody conjugation titrated across multiple RT/ET ratios using fluorophore tracing and gel exclusion chromatography. (FIG. 1E) Fluorescent ET antibody conjugated to liposomes at a range of coating values quantified using area under the curve analysis, demonstrating conjugation across all coating densities (inset graph). (FIG. 1F) Complementary measures of fluorescent RT antibodies of the corresponding liposomes (e.g. 97.5% EC antibody in FIG. 1E and 2.5% RBC antibody in FIG. 1F were read simultaneously from the same liposome sample) analyzed as in FIG. 1E.

FIG. 2A-FIG. 2F show characterization of liposome to RBC binding by RBC/EC antibody conjugation ratio, coating density, and liposome concentration. (FIG. 2A) Agglutination of RBCs by % RBC antibody coating and # liposomes added. Round-bottom well assay demonstrates the effect of % RBC antibody on liposomes and their concentration in which aggregated RBCs appear diffuse and non-aggregated cells settle into a tight red dot. Image data demonstrate the effect of both the RBC v coating density (top left to right, 0-100%) and increased liposome numbers bound (left side top to bottom) affect aggregation of RBCs bound. Human RBCs are tested as a control. RBC samples within the black box define the antibody coating ratios and liposome binding concentration benign to RBC viability with respect to agglutination. (FIG. 2B) ET/DT/RT liposome immunoreactivity shows liposome binding efficiency against a vast excess of RBC binding sites, demonstrating binding to mouse RBCs by % of mouse RBC antibody on particle surface (with the balance of antibody against ICAM). Binding efficiency increases nearly linearly until about 10% RBC mouse antibody, after which binding asymptotically approaches completion. Control binding against human RBCs with the same particles demonstrates maximum potential adsorption of non-RBC-targeted liposomes at a given antibody coating. (FIG. 2C and FIG. 2D) RBC binding to ET/DT/RT liposomes conjugated with EC antibody against ICAM (FIG. 2C) or PECAM (FIG. 2D). RBC liposome in vitro binding by ratio of RBC to EC targeting antibody conjugated to liposome surface (varied ratios at 200 total antibodies/liposome) is proportional to addition, but efficiency of binding is not identical in PECAM vs ICAM. (FIG. 2E) Flow cytometry of RBC loaded with DTRH liposomes and ET-RH liposomes and compared to control (washed) RBC. Flow cytometry was performed on RBC loaded with DTRH liposomes and ET-RH liposomes and compared to control RBC. Liposome-positive RBC demonstrated fluorescence greater than control RBC. Loading RBC with dual-targeted liposomes resulted in 99.7% of the RBC population bound by liposomes, compared to only 73% of the RBC population when loaded with endothelial-targeted RBC. Insets. Fluorescence microscopy of RBC loaded with liposomes. (FIG. 2F) Mean fluorescent intensity (MFI) of the peaks shown indicate 43-fold increase of liposome signal in DTRH vs ET-RH (ET-ICAM).

FIG. 3A-FIG. 3D show biodistribution and RBC-to-organ transfer of ¹²⁵I-liposomes from ⁵¹Cr-RBC varies with RT vs PECAM ET antibody ratios; circulation kinetics dictate localization and magnitude of targeting. Error bars=st dev, n>=4 for all samples. (FIG. 3A) Biodistribution in lung and blood of ¹²⁵I-liposomes in RT-RH (left bars) using 2.5% RT antibody/90% IgG liposomes, ET-RH targeting PECAM (center bars), and DTRH using 2.5% RT/97.5% ET-PECAM ratio. (Values>100% are possible due to the convention of normalizing the signal in an organ by weight in grams; if organ<1 g the result>100%. Localization between DT and ET in the lungs increased>˜2× with the addition of only 5 RBC targeting antibodies; 195 v 200 PECAM Abs. (FIG. 3B)⁵¹Cr-RBC+¹²⁵I liposomes, as described RT ET and DT, tracing identical animals in FIG. 3A, corresponding to identical blood and lungs. Inset in FIG. 3B shows analysis of lung/RBC signal in lung to that of blood, describing numerically the transfer of ¹²⁵I-liposomes from ⁵¹Cr RBCs. (FIG. 3C) Kinetics of DTRH biodistribution of ¹²⁵I liposomes and ⁵¹Cr RBC (inset) in organs from 2-20 min. DT formulation in all bars (striped bars contrasting with light to dark with increased time) is 2.5% RT/97.5% ET-PECAM ratio as shown in FIG. 3A- and FIG. 3B. (FIG. 3D) Immediate rapid high localization of the ¹²⁵I liposome signal in lungs drops levels off by 10 mins, and correspondingly, there is little signal in the blood compartments, with a slight uptake of ¹²⁵I liposomes in the liver, greater in the spleen, which may account for the decreased ¹²⁵I lung signal over time. Comparing the ⁵¹Cr distribution data (inset), the RBC signal increases over time as the labeled RBCs return to the blood pool from the liver and to a lesser extent the lungs, with the spleen data being noisy but largely flat. (FIG. 3D) Comparison of biodistribution 2.5%/97.5% vs 10%/90% RT/ET-PECAM ratio. The increase of the RT antibody from 2.5% (light bars) to 10% (dark bars) with DT with ET PECAM overshadows lung localization of ¹²⁵I in the lung by the 10% formulation. The ⁵¹Cr data (inset) shows that the corresponding signal of RBCs in the lungs that the RBCs did not dissociate and go back to the RBC pool, remained in the lungs; by transfer ratio this equates to 610 vs 120 for the 2.5%/97.5% vs 10%/90% DT data.

FIG. 4A-FIG. 4D show dual-targeted liposomes are modified to target human RBC and endothelial cells and are retained in ex vivo human lung tissue. (FIG. 4A) Characterization of liposome to human RBC binding with liposomes functionalized with (1) ET targeting antibody human PECAM Ab62 and (2) either human RBC antibody CD235a against GPA (left) or Bric69 against Rh (right). The binding of liposomes at varied RT/ET ratios from 100% ET to 100% RT. Left: In vitro binding of CD235a/Ab62 liposomes to RBCs. Inset, assay comparing RBC agglutination in additions of 200-1000 liposomes per RBC for the variety of dual-targeted liposomes indicated. Right: As in left using RBC antibody Bric69 against Rh. Inset, agglutination assay as previously described. (FIG. 4B) Schematic of ex vivo lung perfusion (EVLP). The pulmonary artery is cannulated then sample is perfused into the pulmonary artery; it continues through the pulmonary vascular network and ultimately out the pulmonary vein where efflux is collected. (FIG. 4C) Fresh human lung tissue prepared for EVLP. Both right upper lobe (RUL) and right middle lobe (RML) bronchi are cannulated for inflation. Pulmonary artery is cannulated for perfusion. Green tissue dye is perfused to confirm adequate cannulation and perfusion through the vasculature with efflux seen oozing from the pulmonary vein. (FIG. 4D) Ex vivo human lungs were perfused using DTRH humanized liposomes. Liposomes were traced with ¹²⁵I and RBC were traced with ⁵¹Cr. Perfused portion of lung tissue was visualized using green tissue dye, then dissected and retention of liposomes and RBC measured. Of initial injected dose, 27.5% remained in the lung tissue after perfusion compared to only 15.4% of carrier RBC.

FIG. 5A-FIG. 5D show biodistribution and pharmacokinetics of RH delivery of RT, ET and DT using ICAM endothelial targeting versus equivalent free liposomes. (FIG. 5A and FIG. 5B) Biodistribution of RH delivery of liposomes RT, ET, and DT at 10% and 25% RBC antibody coating (v 90% and 75% ICAM respectively), ex vivo loaded onto ⁵¹Cr labeled RBCs (FIG. 5A) with ¹²⁵I labeled RT, ET and DT distributions (FIG. 5B). Tracing the ¹²⁵I-liposomes in the lung data shows accumulation of the 10% DT sample is more than double that of the ET sample, but is significantly diminished in the 25% sample. Higher values in liver and spleen in the 25% DT v 10% DT account for the difference. Comparison of the RBC data to that of the lung indicates that carrier RBCs are retained in circulation, not in the lungs. (FIG. 5C) Free liposome delivery (non-RBC bound) of ¹²⁵I labeled samples identical to those in FIG. 5B. Variation in lung targeting of directly injected liposomes containing ET targeted antibody varies slightly, proportionally to the extent of ICAM coating (200 vs 180 vs 150 in #/liposome of ICAM antibody of ET vs DT 10%/90% vs DT 25%/75%). Clearance organs liver and spleen showed similar accumulation across the different direct injection samples, and little association with blood. (FIG. 5D) Pharmacokinetics comparing endothelial, dual, and RBC targeted liposomes delivered by RBC versus free injection. Data organized as described in the inset table left side of FIG. 5D. Group 1 (DT) represents 10% RT/90% ET ICAM, group 2 (ET) data are 0% RT Ab/100% ET ICAM, and group 3 (RT) data are 100% RT/0% ET ICAM. PK time points 5, 10, and 20 min (in each bar grouping of 3, left to right with time increasing) post IV injection. Y-axis scales of graphs are consistent left to right. Right column graphs show localization to organs by injected dose per gram of designated organ tissue (% ID/g) over time of the freely injected liposomes without RBCs (as in FIG. 5C), the left and center column data represent ¹²⁵I and ⁵¹Cr on RBCs in the same animal's organs using liposomes identical to those in the right (free liposome) column. DTRH vs free DT liposome Group 1—liposomes have 20 RT/ET (ICAM) #/liposome and tracer ¹²⁵I-IgG, bound to ⁵¹Cr labeled equivalently to other groups for each graph. The inset center graph emphasizes the reduction of RBC ⁵¹Cr signal in the lung over time as the ⁵¹Cr RBCs separate from their ¹²⁵I liposome cargo, whereas the corresponding ¹²⁵I-liposome signal in the left graph is unchanged. Comparing the lung data in the right column graph of the freely injected DT liposomes shows that localization in the lungs of the left column graph DTRH liposomes is enhanced with a ˜2.5-fold increase. Group 2—data of ET freely injected liposomes (right column) compared to ET-RH (left column) shows that there is an enhancement with RH delivery, p<0.05 of the ¹²⁵I signal between the localization in the lung even without dual targeting. Group 3-RT liposomes are traced as in groups 1 & 2. RT directly injected vs RT-RH, are mainly cleared in liver and spleen with slight and diminishing accumulation in the lungs. Data show that liposomes with a high concentration of RBC antibodies are immediately sequestered to clearance organs, liver and spleen, rather than enduring bound and circulating to RBCs, as shown by low signal in blood components. All data represented have n>=4, error bars are standard deviation.

FIG. 6A-FIG. 6E show dual-targeted RBC hitchhiking (DTRH) enhances the selectivity of targeted binding to specific cell types of interest. (FIG. 6A and FIG. 6B) Flow cytometry of single cell suspensions of lung tissue showing increased targeting to endothelial cells and leukocytes with DTRH ICAM of 10% RBC Ab. (FIG. 6C) Fluorescence micrographs indicating association of liposomes (red) with either endothelial cells (left) or leukocytes (right) in the lung after circulation for 30 minutes. (FIG. 6D) Quantification of flow data showing percent of two cell populations, endothelial cells and leukocytes, bound by liposomes using DTRH versus equivalent DT freely injected liposomes. Quantification of flow cytometry data indicates that DTRH results in a 20-fold increase in endothelial cell targeting and near 4-fold increase in leukocyte targeting. (FIG. 6E) Analysis of cell localization of DTRH vs freely injected liposomes. Left bar indicates the increase in endothelial cell localization by fold increase from free injection to DTRH and the right bar an equivalent calculation of leukocyte localization. Cell type specification shows a 6.4× fold increase of endothelial cells vs leukocytes.

FIG. 7 shows gel exclusion chromatography elution profiles of varying amounts of % RBC antibody on DT liposomes traced by 125I-IgG-DBCO. Half ml fractions are collected as elution volume versus normalized % CPM of the entire elution. Extent of conjugation of antibodies to liposomes calculated by area under the curve (AUC) of the liposome peak at 5.5 ml to 7.5 ml relative to the AUC of the whole elution. Efficiency of conjugation is >=90-98% of total measured, which was consistent across the varied ratios of DT liposomes studied.

FIG. 8A-FIG. 8B show effects of RBC antibody coating density and liposome concentration on RBC binding, biocompatibility, safety. (FIG. 8A) Binding efficiency of DT liposomes coating density, time, and #liposomes added per RBC. % RBC expressed per 200 total antibodies added/liposome with ICAM making up the balance. A small but significant increase in binding efficiency was seen in the increase of binding time from 90 mins to overnight (˜16 h) in the lower % coated liposomes, and little change was seen between 200 liposomes and 500 added/RBC. The higher coating concentration formulations at the lower numbers of liposomes/RBC (10 & 100%; solid bars) showed near complete binding to RBCs and little difference between them, but there was a predictable decrease in efficiency at the shorter time with fewer liposomes added. (FIG. 8B) Agglutination assay (as described in FIG. 2A) of binding efficiency samples in graph. Samples are in replicates. Agglutination, as defined by the spreading of the cell pellet as RBCs aggregate, is observed in every concentration of the 100% RBC antibody coated DT liposomes added (bottom row). Of the lower % RBC antibody coated DT-liposomes, only the higher liposome concentration overnight in the 10% sample showed some agglutination. These data establish boundaries of % RBC antibody coating and liposome binding concentrations.

FIG. 9 shows a table for evaluation of variability by RBC source and DT liposome preparation.

FIG. 10A-FIG. 10B show DTRH yields better binding of liposomes to RBC compared to ET-RH. (FIG. 10A) DTRH liposomes bind to RBC better than passive RBC hitchhiking (RH) liposomes. DTRH liposomes were coated with anti-RBC antibodies and anti-endothelial cell antibodies. ET liposomes were coated with anti-endothelial cell antibodies. Liposomes were loaded onto RBC using our standard protocol (90 min, gentle rotation). When loaded with dual-targeted liposomes, 99.7% of RBC population is bound by liposomes. When loaded with passive liposomes not targeted specifically to the RBC, only 73% of RBC population is bound by liposomes, center. (FIG. 10B) Similar methods as in FIG. 10A; fluorescent DTRH liposomes were bound to RBC and imaged using confocal microscopy. Representative images reveal more homogenous and brighter liposome coating on DTRH-loaded RBC.

FIG. 11 shows biodistribution data varies with the extent of liposomal antibody coating at 10% RT/90% non-immune IgG at low-, mid-, and high-density coating. Biodistribution of ¹²⁵I liposomes and ⁵¹Cr-RBCs (inset) in RT-RH at 10% RT Ab/90% non-immune control IgG coated liposomes at increasing antibody density in designated organs. (Solid bar data in inset represents unmodified RBC controls with no liposomes.) Striped bars with increasing white to black contrast indicate increasing density of coating antibodies of 25, 100 and 200 total number, maintaining a constant RT:IgG antibody ratio. Decreasing overall antibody coating density allows increased retention in RBC blood pool, and decreased clearance into liver and spleen. Correspondingly the ⁵¹Cr data show higher clearance of RBC into spleen with higher antibody coating on liposomes, decrease of blood circulation. Error bars represent standard deviation, n>=4 for all samples.

FIG. 12A-FIG. 12C show organ transfer ratios of RT, ET and DTRH with PECAM of biodistribution and pharmacokinetics. (FIG. 12A) Schematic of liposomal and lung transfer ratios. Top schematic of the transfer of the liposomes from the blood compartment to the lung over time as described by the liposome ratio of % ID/g ¹²⁵I lung to % ID/g ¹²⁵I RBC. Bottom schematic represents the lung ratio which illustrates the transfer of the particle from the carrier RBC within the lung using the ratio of signals ¹²⁵I:⁵¹Cr in the lung. B&C) The graphs on the right enumerate both ratios working together derived from PECAM data of FIG. 3A-FIG. 3D. (FIG. 12B) Graphical representation of ratio of liposome ratio compared to lung ratio derived from data shown in FIG. 2C and FIG. 2D. The greatly enhanced liposome ratio of DT 10%/90% shows a simultaneously diminished lung ratio, indicating high localization of liposomes but poor RBC transfer. Although DT 2.5%/97.5% has a moderate liposome ratio, the lung ratio is maximal across the samples studied. (FIG. 12C) Summary of particle transfer by analysis of DTRH PK data. Graph of liposome ratio compared to lung ratio derived from data shown in FIG. 3C. The particle ratio data of the bar graph indicate that the signal of ¹²⁵I-liposomes localize in the lung immediately and predominantly versus in the RBC compartment. The overlaid line graph shows between the two isotope signals in the lung the particle signal is dominant and increasing over time versus the RBC ⁵¹Cr signal.

FIG. 13 shows transfer ratios DTRH with ICAM pharmacokinetics. Graphical representation of ratio of liposome ratio compared to lung ratio derived from data shown in FIG. 4B and FIG. 4C. The graph enumerates the liposome and lung ratios working together. The liposome ratio data of the bar graph indicate that the signal of ¹²⁵I-liposomes localize in the lung immediately and predominantly versus in the RBC compartment. The overlaid line graph shows between the two isotope signals in the lung the particle signal is dominant and increasing over time versus the RBC ⁵¹Cr signal.

FIG. 14 shows biodistribution at 30 minutes after intravenous injection in a murine model. DT-liposomes are labeled with ¹¹¹In for tracing. DT-liposomes were loaded onto RBC by drawing whole blood into a syringe containing ICAM DT-liposomes (Ter119=RT Ab) then waiting for 10 or 30 minutes for loading. These were compared to injection of free DT-liposomes (solid) or standard ex vivo RBC-loaded DTRH (striped).

### DETAILED DESCRIPTION

A significant number of drugs have serious side effects and toxicities that limit their use. The field of nanomedicine has long promised solutions to this problem, and several nanomedicines are available clinically. However, despite the previously demonstrated drug-carrying capacity of targeted nanocarriers, and accepted clinical use, several challenges remain. These include rapid clearance from circulation by the reticuloendothelial system (RES) and uptake by circulating immune cells. Described herein are targeted nanoparticles (e.g., liposomes) that utilize RBC-hitchhiking to improve drug delivery to target organs.

Red blood cells have been used as drug shuttles and carriers in prior work and in combination with nanocarriers, termed RBC hitchhiking. This first generation of RBC hitchhiking, in which a nanocarrier is associated with an RBC by nonspecific binding, has been shown to successfully evade nanocarrier uptake by the RES. However, these first-generation RBC-nanocarrier complexes had limitations. Hitchhiked particles were non-liposome nanocarriers and thus had reduced potential for clinical development and increased potential to cause damage to the carrier RBC. In some cases, non-liposome nanocarriers were observed to cause toxicity in their RBC shuttles. Additional studies have advanced the theory of RBC-hitchhiking into a reality. RBC were shown to be passively loaded with a variety of nanocarriers including liposomes that enabled targeted delivery to the lungs at an order of magnitude higher than previously obtained with targeted nanocarriers. This lung targeting was shown to be safe in the lung vasculature by using pulmonary artery pressure waveforms. Despite this recent advancement in the field, several critical areas of RBC-hitchhiking need improvement.

Previous RBC-hitchhiking research has relied in some cases on nonspecific adsorption of particles onto RBC. This nonspecific hitchhiking presents several challenges. First, there is no control of binding to the RBC and therefore the association of a particle to RBC is not able to be manipulated for different scenarios. Second, delivery to target organs likely still relies heavily on first pass delivery since the particle can readily separate from the RBC. Third, obtaining nonspecific binding is time consuming and prone to variability. Finally, while new particles may be engineered to nonspecifically adhere to RBC, this technique would exclude all other existing drugs and those in development.

An alternative targeting method that has been previously explored is cell-based delivery of nanocarriers. A popular cell-based delivery methodology has been loading nanocarriers ex vivo into leukocytes, usually via phagocytosis. Upon injection, the nanocarrier-loaded leukocytes travel to destination tissues.⁹⁻¹¹ Since leukocytes can behave unpredictably, and are not currently isolated in this manner clinically, red blood cells (RBCs) provide a simpler, cell-based delivery option.

Dual-targeted RBC hitchhiking (DTRH) actively targets nanoparticles to RBC to overcome many of the limitations of passive RBC-hitchhiking. For example, liposomes are engineered to display RBC-targeting and organ-specific moieties on their surfaces, allowing precise control of the association between liposomes and RBC. This controlled loading does not cause harm to the carrier RBC. In some cases, the surface of dual-targeted liposomes replaces 10% of the tissue targeting moieties on a liposome with RBC-targeting moieties, which was shown to yield a 2.5 times greater dose delivery to the lungs compared to a single-targeted particle, and 70% of total injected dose is delivered to the lungs. In addition to unprecedented dose delivery to the lungs, dual-targeted liposomes have been shown to transfer from carrier RBCs in vivo. Thus, while antibody targeting has been demonstrated previously, dual-targeted liposomes can sequentially target two different cell types. Such two-cell targeting exploits the myriad interactions that different cell types undergo together, potentially enabling nanocarrier transfer from one cell to another with precise timing and specificity.

RBCs possess multiple advantages as cellular carriers, and their transporting function can be co-opted to shuttle nanocarriers throughout the circulation. They are standardly isolated clinically, have a long shelf-life, and have well understood donor-recipient compatibility. RBCs collectively comprise the largest cellular surface area, and have a months-long blood half-life.

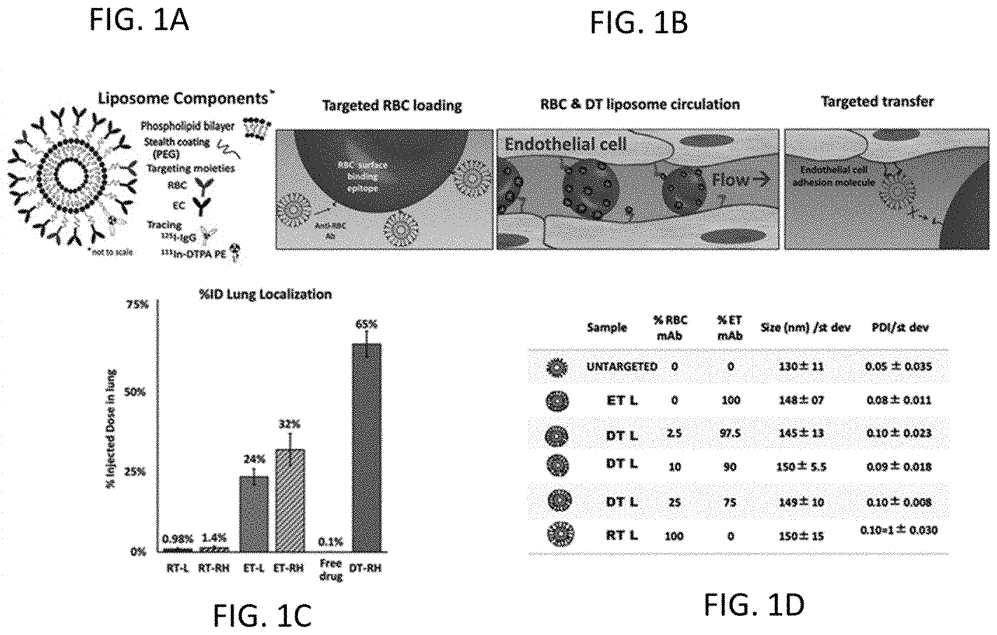

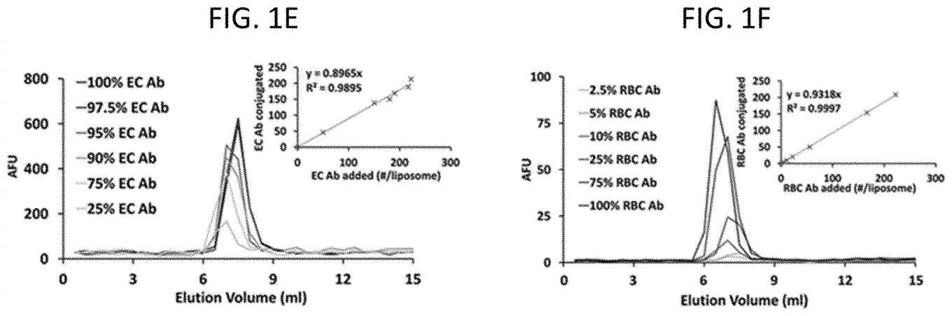

Thus, the compositions and methods provided herein take advantage of the improved drug delivery obtained using RH, and further enhance specificity and flexibility by using ligand-mediated targeting. Termed “dual-targeted RBC hitchhiking” (DTRH), nanocarriers are conjugated with two distinct ligands that bind two different cell types: RBCs and target endothelial cells (FIG. 1A). This approach eclipses earlier versions of RH, by which select nanocarriers were passively and nonspecifically adsorbed onto RBC without the use of RBC-targeting ligands. The passive RH adsorption process has several deficits: i) the inability to work consistently or efficiently with the vast majority of liposomes, the most prevalent, biocompatible nanocarrier; ii) inefficient passive adsorption, usually with <5-10% of the nanocarrier adsorbing onto RBCs; iii) poorly controlled cellular binding affinities, limiting the ability to iteratively improve the technology; iv) poor cellular specificity, targeting not just to endothelial cells, but also to leukocytes in the downstream organ. By adding ligand-targeting, DTRH solves these problems while providing better total organ uptake.

Provided herein are compositions comprising dual-targeted nanoparticles and uses therefor, wherein the dual-targeted nanoparticles have a first targeting moiety specific for a red blood cell (RBC) and a second-targeting moiety. In certain embodiments, provided are compositions having dual-targeted nanoparticles having a first targeting moiety bound to RBCs ex vivo and a second-targeting moiety for administration to a subject. In certain embodiments, the first and/or second targeting moiety is an antibody or an antibody fragment, a carbohydrate, or a carbohydrate-binding compound.

As a prototype for DTRH, the inventors focused on the largest cell-cell interface in the body, RBCs with pulmonary endothelial cells. Specifically, carrier RBCs loaded with liposomes shuttle through the circulation until contact with, and transfer to, the pulmonary endothelium (FIG. 1B). RBCs serve as an intermediary carrier with the liposome transiently bound to its surface. The results showed that the relative avidities of dual-targeted liposomes for RBC versus endothelial cells can be modified to enhance the precision of delivery to target cells. They showed that dual-targeted liposomes can be engineered to safely, efficiently, and predictably bind RBCs without inducing aggregation. Optimized DTRH liposomes more than doubled delivery to the lungs in vivo compared to standard endothelial-targeted liposomes, finally achieving a majority (>50% of the injected dose) of the nanocarrier going to the target organ. Enhanced delivery can be achieved using various endothelial targets, including ICAM and PECAM. Further, DTRH also markedly improve cell-type-specific targeting, increases the ratio of endothelial to non-endothelial cells taking up nanocarriers in the target organ.

By “nanoparticle” or “NP” (also referred to as “nanocarrier” or “NC”) as used herein is meant a particle having diameter of between about 1 to about 1000 nm. In one embodiment the NP is globular. Inclusive in this definition are particles with a diameter of at least 1, at least 20, at least 40, at least 60, at least 80, at least 100, at least 120, at least 140, at least 160, at least 180, at least 200, at least 220, at least 240, at least 260, at least 280, at least 300 nm in diameter. In other embodiments, also included are particles having diameters of at least 320, at least 340, at least 360, at least 380, at least 400, at least 420, at least 440, at least 460, at least 480, at least 500, at least 520, at least 540, at least 560, at least 580, at least 600, at least 620, at least 640, at least 660, at least 680, or at least 700 nm. In yet other embodiment, also included are particles having diameters of at least 720, at least 740, at least 760, at least 780, at least 800, at least 820, at least 840, at least 860, at least 880, at least 900, at least 920, at least 940, at least 960, at least 980, or up to about 1000 nm. All numbers and fractions between any two of these numbers are also included. In certain embodiments, a dual-targeted NP is provided having a diameter of about 10 nm to about 1000 nm. In yet a further embodiment a dual-targeted NP is provided having a diameter of about 145 nm to about 150 nm.

In one embodiment, the nanoparticle (NP) is a liposome. By “liposome” as used herein is meant a material a microscopic spherical particle formed by a lipid bilayer enclosing an aqueous compartment. In certain embodiments, the nanoparticle is a hydrogel NP (also referred to as a nanogel or NG). In certain embodiments, the NP is a dendrimer. In yet other embodiments, the NP is a polymersome, a hybrid carrier combining artificial and natural components, or a protein multimolecular composition (e.g., ferritin or albumin aggregates).

By “hydrogel nanoparticle” or “nanogel” as used herein is mean a polymeric material having a hydrophilic structure which renders it capable of holding amounts of selected drug compounds in their three-dimensional networks, the resulting particle having nanoparticle dimensions. Macroscopic dextran hydrogels have shown Young's moduli of ˜10-50 kPa in the literature. See, e.g., Hwang M R, Kim J O, Lee J H, Kim Y I, Kim J H, Chang S W, Jin S G, Kim J A, Lyoo W S, Han S S, Ku S K, Yong C S, Choi H G. Gentamicin-Loaded Wound Dressing with Polyvinyl Alcohol/Dextran Hydrogel: Gel Characterization and In vivo Healing Evaluation. AAPS PharmSciTech 2010; 11(3):1092-1103. In certain embodiments, the NG is a lysozyme-dextran nanogel (also referred to as LDNG). In certain embodiments, the LDNG is a synthetic construct of lysozyme and dextran. In other embodiments, the nanogel is made of chitosan or chitin, pullulan, hyaluronic acid, PEG, pluronics (e.g. F127), poly(acrylic acid) or poly(acrylate), poly(oligo(ethylene glycol)methyl ether methacrylate), poly(ethylene oxide), polyethylenimine, poly(caprolactone), and poly(N-isopropylacrylamide), among other options encompassing a wide range of hydrophilic polymers capable of chemical modifications enabling incorporation in a nanoparticle. See, e.g., Eckmann D M, Composto R J, Tsourkas A, Muzykantov V R. Nanogel Carrier Design for Targeted Drug Delivery. J Mater Chem B Mater Biol Med 2015; 2(46):8085-8097; and Ahmed E M, March 2015, “Hydrogel: Preparation, Characterization, and Applications: A Review”, J. Adv. Res., 6(2):105-121, among other publications in the art. In certain embodiments described herein, nanogel particles containing a drug and associated with an RBC behave best in this form of drug delivery.

In certain embodiments, the nanogel or NP has a carbohydrate surface which aids with RBC and endothelial glycocalyx interaction. In another embodiment, the protein coating, e.g., IgG or albumin, prevents RBC toxicity. In yet another embodiment, the protein-coated nanogels are cross-linked or lyophilized. In still another embodiment, the nanogel or flexible NP has a capacity sufficient for large drugs or imaging agents. In certain embodiments, the nanoparticle has a polyethylene glycol (PEG) coating. In yet a further embodiment, the nanoparticle is a biological particle (e.g. an exosome or LDL).

The dual-targeted nanoparticles for use in the compositions and methods described herein have at least one targeting moiety that specifically binds a RBC surface antigen. In certain embodiments, the nanoparticles have a targeting moiety specific for a RBC target antigen that is a glycophorin, optionally glycophorin A (GPA) or Band 3. In certain embodiments, the nanoparticles have a targeting moiety specific for an Rh antigen. In certain embodiments, the nanoparticles have a targeting moiety specific for RhCE. Iin certain embodiments, the targeting moiety that specifically binds a RBC surface antigen is the CD235a monoclonal antibody or the Bric69 monoclonal antibody.

By the term “targeting moiety” as used herein, is meant a molecule, including an antibody, a fragment thereof or an antibody fusion protein, which is capable of specifically binding to another molecule. A targeting moiety may be an antibody, an aptamer, a nucleic acid, a peptide, a carbohydrate (sugar), a lipid, a vitamin, a toxin, a component of a microorganism, a hormone, a receptor ligand, and or any derivative thereof. If the targeting moiety is an antibody, it may be a monoclonal antibody, a humanized antibody, a synthetic antibody, a heavy chain antibody, and a biologically active fragment of an antibody, wherein the biologically active fragment is a Fab fragment, a F(ab′)₂ fragment, and a Fv fragment.

As provided herein, a number of parameters contribute to improved delivery of nanoparticles using the compositions provided herein, including, but not limited to the total number of targeting moieties and the relative abundance of first and second targeting moieties. In certain embodiments, the dual-targeted nanoparticles are characterized by having about 5 to about 350 of a first targeting moiety per nanoparticle. In certain embodiments, the dual-targeted nanoparticles are characterized by having at least 5, at least 20, at least 50, at least 75, at least 100, at least 100, at least 150, at least 200, at least 250, at least 300, or at least 350 first targeting moieties per nanoparticle. In yet another embodiment, the dual-targeted nanoparticle is characterized by having less than 5, less than 20, less than 50, less than 75, less than 100, less than 100, less than 150, less than 200, less than 250, less than 300, or less than 350 first targeting moieties per nanoparticle. In yet another embodiment, the total number of first and second targeting moieties is about 5 to about 350 per nanoparticle. In certain embodiments, the dual-targeted nanoparticles are characterized by having a total of at least 5, at least 20, at least 50, at least 75, at least 100, at least 100, at least 150, at least 200, at least 250, at least 300, or at least 350 first and second targeting moieties per nanoparticle. In yet another embodiment, the dual-targeted nanoparticles are characterized by having a total of less than 5, less than 20, less than 50, less than 75, less than 100, less than 100, less than 150, less than 200, less than 250, less than 300, or less than 350 first and second targeting moieties per nanoparticle.

In certain embodiments, the dual-targeted nanoparticles have a specific proportion of first and second targeting moieties. As provided herein, the first targeting moiety (RBC-specific) may range from about 2.5% to about 50% of total targeting moieties of the dual-targeted nanoparticle. In certain embodiments, the first target moiety is at least 2%, at least 2.5%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% of total targeting moieties of the dual-targeted nanoparticle. In yet another embodiment, the first targeting moiety is less than 2%, less than 5%, less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, less than 85%, less than 90% of total targeting moieties of the dual-targeted nanoparticle. All numbers and fractions or ratios between any two of these percentages are also included, as well as endpoints.

As described herein, the number of nanoparticles bound to an RBC can alter the preparation of a composition (e.g. reduce agglutination ex vivo, limit disruption of RBC membranes, and improve loading of RBCs with nanoparticles) and levels of drug delivery to one or more target organs. Accordingly, in certain embodiments, provided herein are compositions comprising dual-targeted nanoparticles having RBCs bound ex vivo, wherein the composition has about 50 to about 200 bound nanoparticles per RBC. In certain embodiments, the composition has at least 2, at least 5, at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, or at least 200 bound nanoparticles per RBC. In yet another embodiment, the composition includes of less than 5, less than 10, less than 20, less than 30, less than 40, less than 50, less than 70, less than 80, less than 90, less than 100, or less than 110, less than 120, less than 130, less than 140, less than 150, less than 160, less than 170, less than 180, less than 190, or less than 200 bound nanoparticles per RBC. All numbers and ranges between any two of these numbers are also included, as well as endpoints.

In certain embodiments, a nanoparticle composition is provided which comprises dual-targeted nanoparticles having about 10% to about 25% of a first RBC-targeting moiety and about 75% to about 90% of a second targeting moiety that binds, e.g., an endothelial target.

In certain embodiments, a nanoparticle composition is provided which comprises dual-targeted nanoparticles having about 2.5% to about 5% of a first RBC-targeting moiety and about 95% to about 97.5% of a second targeting moiety that binds, e.g., an endothelial target.

In certain embodiments, a nanoparticle composition is provided which comprises dual-targeted nanoparticles having about 2.5% to about 10% of a first RBC-targeting moiety and about 90% to about 97.5% of a second targeting moiety that binds, e.g., an endothelial target.

All of the ranges described herein include endpoints.

By the term “antibody” or “antibody molecule” is any immunoglobulin, including antibodies and fragments thereof, that binds to a specific antigen. As used herein, antibody or antibody molecule contemplates intact immunoglobulin molecules, immunologically active portions of an immunoglobulin molecule, and fusions of immunologically active portions of an immunoglobulin molecule.

The antibody may be a naturally occurring antibody or may be a synthetic or modified antibody (e.g., a recombinantly generated antibody; a chimeric antibody; a bispecific antibody; a humanized antibody; a camelid antibody; and the like). The antibody may comprise at least one purification tag. In a particular embodiment, the framework antibody is an antibody fragment. The term “antibody fragment” includes a portion of an antibody that is an antigen binding fragment or single chains thereof. An antibody fragment can be a synthetically or genetically engineered polypeptide. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment, which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv). Such single chain antibodies are also intended to be encompassed within the term “antigen-binding fragment” of an antibody. These antibody fragments are obtained using conventional techniques known to those in the art, and the fragments can be screened for utility in the same manner as whole antibodies. Antibody fragments include, without limitation, immunoglobulin fragments including, without limitation: single domain (Dab; e.g., single variable light or heavy chain domain), Fab, Fab′, F(ab′)₂, and F(v); and fusions (e.g., via a linker) of these immunoglobulin fragments including, without limitation: scFv, scFv2, scFv-Fc, minibody, diabody, triabody, and tetrabody. The antibody may also be a protein (e.g., a fusion protein) comprising at least one antibody or antibody fragment.

As used herein, “specifically binding,” “binds specifically to,” “specific binding” refer, for example, to an antibody selectively or preferentially binding to an antigen. For example, with respect to a targeting moiety (such as an antibody), specifically binding refers to preferential binding refers to the ability of the antibody to bind one or more epitopes of an antigen or binding partner of interest without substantially recognizing and binding other molecules in a sample or environment containing a mixed population of antigens. Specific binding interactions are mediated by one or, typically, more noncovalent bonds between the binding molecules or binding partners.

The antibodies utilized herein may be further modified. For example, the antibodies may be humanized. In a particular embodiment, the antibodies (or a portion thereof) are inserted into the backbone of an antibody or antibody fragment construct. For example, the variable light domain and/or variable heavy domain of the antibodies of the instant invention may be inserted into another antibody construct. Methods for recombinantly producing antibodies are well-known in the art. Indeed, commercial vectors for certain antibody and antibody fragment constructs are available.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a nonhuman species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence, except for FR substitution(s) as noted above. The humanized antibody optionally also will comprise at least a portion of an immunoglobulin constant region, typically that of a human immunoglobulin. For further details, see Jones et al, Nature 321:522-525 (1986); Riechmann et al, Nature 332:323-329 (1988); and Presta, Curr. Op. Struct. Biol 2:593-596 (1992).

The term “antibody fragment” as used herein for the described methods and compositions refers to less than an intact antibody structure having antigen-binding ability. Examples of antibody fragments include Fab, Fab′, F(ab′)2, and Fv fragments; diabodies; linear antibodies; single-chain antibody molecules such as e.g. single chain Fab, scFv, and multispecific antibodies formed from antibody fragments. The “single chain Fab” format is described, e.g., in Hust M. et al. BMC Biotechnol. 2007 March 8; 7:14. scFvV constructs include complementary scFvs produced as a single chain (tandem scFvs) or bispecific tandem scFvs.

In certain embodiments, the nanoparticle compositions provided herein include a second targeting moiety that is selected for delivery to, e.g, a target organ or tissue, such as antibody that bind to the endothelium (e.g., antibodies targeting endothelial proteins including PECAM-1, ICAM-1, VCAM, ACE, transferrin receptor, tissue factor/platelet tissue factor/factor III, and others) or antibodies that bind to other targeted cells. The targeting moiety may bind to at least one of a cell surface protein, carbohydrate, or lipid. In some embodiments, the targeting moiety binds to a cell adhesion molecule (CAM). The CAM may be intercellular adhesion molecule (ICAM), platelet-endothelial cell adhesion molecule (PECAM), activated leukocyte cell adhesion molecule (ALCAM), B-lymphocyte cell adhesion molecule (BL-CAM), vascular cell adhesion molecule (VCAM), mucosal vascular addressin cell adhesion molecule (MAdCAM), CD44, LFA-2, LFA-3, P-selectin, and basigin. In certain embodiments, the targeting moiety binds specifically to a CAM selected from ICAM, PECAM, or VECAM. In certain embodiments, the nanoparticles include a second targeting moiety specific for ICAM-1 and are targeted to pulmonary endothelial cells and white blood cells in the lungs (since ICAM-1 is expressed on both cell types). In yet other embodiments, the targeting moiety binds specifically to ACE, transferrin receptor, or a suitable endothelial target known in the art (See e.g., Simone et al. Cell Tissue Res. 2009 January; 335(1): 283-300, which is incorporated herein by reference). In other embodiments, the targeting moiety binds to a cell surface molecule associated with classical endocytosis. Preferably, the cell surface molecule associated with classical endocytosis is one of mannose-6-phosphate receptor and transferrin receptor. In certain embodiments, the second targeting moiety is specific for one or more of vascular endothelial cells, intravascular leukocytes, cells of reticuloendothelial system, an immune cell (e.g., B cell, T cell, NK cell, dendritic cell, macrophage), an infectious microorganism (including, but not limited to, a viral particle or bacterium), or targets tissues accessible to RBC under pathological conditions such as hemorrhage and thrombosis.

The surface of the NP may be modified to facilitate insertion, conjugation, absorption, or otherwise coupling of a moiety, either directly or via a spacer. The first and/or second targeting moieties of the invention may be linked noncovalently or covalently to the nanoparticles. Covalent linkages include linkages susceptible to cleavage once internalized in a cell. Such linkages include pH-labile, photo-labile and radio-labile bonds and are well known in the art. A targeting moiety may be directly or indirectly linked to a nanoparticle, e.g. via an avidin- or streptavidin-biotin interaction. In certain embodiments, the compositions include nanoparticles (e.g. liposomes) having a polyethylene glycol coating that includes or mediates attachment of a targeting moiety to the nanoparticle. In certain embodiments, the nanoparticles are chemically conjugated to targeting moieties using molecular cross-linkers, spacers, and bridges. By cross-linkers, spacer and bridges are meant any moiety used to attach or associate the NP to the targeting moiety. Thus, in one embodiment, the cross-linker is a covalent bond. In another embodiment, the linker is a non-covalent bond. In still other embodiments, the linker can be a larger compound or two or more compounds that associate covalently or non-covalently. In still other embodiment, the linker can be a combination of the linkers, e.g., chemical compounds, nucleotides, amino acids, proteins, etc. In one embodiment, the cross-linker is biotin-avidin or biotin-streptavidin. In this embodiment, interconnecting molecule(s) such as streptavidin can be coupled to a targeting moiety or an RBC either directly via chemical modification, or via biotin derivatives conjugated to the functional groups on the targeting moiety. In one embodiment a spacer is positioned between biotin and a reactive group, such as succinimide ester group. In certain embodiments, a spacer version is a polyethylene glycol (PEG) chain with MW from ˜100 Daltons (D) to up to 10,000 D. In another embodiment, the spacer is an aliphatic chain —CH₂—CH₂—CH₂— with size varying from 1 angstroms (A) up to 5, 10, 15, 20, 25 or 30 Angstroms. Longer spacer arms give more flexibility for interactions. In another embodiment the linker is composed of at least one to about 25 atoms. In still another embodiment, the cross-linker is formed of a sequence of at least 2 to 60 nucleic acids. In yet another embodiment, the cross-linker refers to at least one to at least 2, up to about 30 amino acids or 1 or more proteins within that size. In certain embodiments, the linkage of the first and/or second moiety to the nanoparticles is reversible (e.g. following administration to the subject and binding of the second moiety to a target antigen).

In certain embodiments, the NPs for use in this invention are pre-loaded with a cargo (e.g. before coupling to a RBC the NP is loaded with one or more selected drugs). The drug may be encapsulated in the inner volume and/or bound to the surface of nanoparticles. In one embodiment, the NP loading is high capacity, e.g., the mass of the drug is >5% the mass of the NP. In one embodiment the selected NP contains a single drug component. In another embodiment, the selected NP is loaded with multiple drug components. By “drug” as used herein is meant any therapeutic, prophylactic, or diagnostic compound or reagent that is contained within the flexible nanoparticles described herein. In one embodiment, the drug is a water-miscible compound. In another embodiment, the drug is an anti-rejection drug. Anti-rejection drugs include agents such as alemtuzumab, tacrolimus, and other drugs currently delivered systemically post-transplant to prevent rejection. In another embodiment, the drug is an anti-inflammatory agent. Anti-inflammatory agents include corticosteroids, methotrexate, mycophenolate mofetil, azathioprine and other agents intended to limit inflammation. In another embodiment, the drug is a pro-angiogenic factor, such as VEGF-receptor agonists and other known such factors. In another embodiment, the drug is an anti-edema agent, e.g., albuterol. In still another embodiment the drug is a compound that prevent ischemia-reperfusion injury, such as N-acetylcysteine, allopurinol, L-arginine, among known agents.

In a specific example, where the composition is designed for the improvement of ARDS, in one embodiment, multiple drugs are employed in the NPs. These drugs include combinations that act on multiple cell types, such as albuterol (acting on epithelial ENac to pump out alveolar fluid), dexamethasone (enhances endothelial barrier function and decreases neutrophil activity), and palifermin (enhances repair and regeneration of alveoli). Other drug combinations with possible applications in other diseases include but are not limited to: Imatinib Mesylate (trade name Gleevec, a tyrosine kinase inhibitor approved for treatment of a variety of cancers), EUK-134 (a superoxide dismutase and catalase mimetic capable of mediating oxidative stress injury), MJ33 (an NADPH oxidase inhibitor used for antioxidant protection). Still other drug(s) or combinations for loading in the NPs of these compositions may be selected from among libraries of drugs for a variety of diseases.

In still another embodiment, the drug is an imaging agent. An “imaging agent” as used herein is a compound that has one or more properties that permit its presence and/or location to be detected directly or indirectly. Examples of such imaging agents include proteins and small molecule compounds incorporating a labeled entity that permits detection. Among suitable imaging agents are molecules containing radionuclides that are amenable to SPECT or PET imaging (e.g., Indium-111 for SPECT imaging); molecules containing moieties that provide contrast for CT imaging (e.g., gold nanoparticles or iodinated contrast agents); molecules containing moieties that provide contrast for MRI imaging (e.g., gadolinium); nano- or micro-scale complexes that provide contrast for ultrasound imaging (e.g., microbubbles filled with gas). A “detectable label” is a marker used for detection or imaging. Examples of such labels include: a radiolabel, a fluorophore, a chromophore, or an affinity tag. In one embodiment, the label is a radiolabel used for medical imaging, for example tc99m or iodine-123, or a spin label for nuclear magnetic resonance (NMR) imaging (also known as magnetic resonance imaging, MRI), such as iodine-123, iodine-131, indium-111, fluorine-19, carbon-13, nitrogen-15, oxygen-17, gadolinium, manganese, iron, etc.

In another aspect, a suitable nanoparticle for use in the methods herein is miscible with endothelial glycocalyx (e.g., carbohydrates on nanogel interdigitate with endothelial cells).

In another aspect, a suitable nanoparticle for use in the methods herein is characterized by having a more efficient or effective delivery than previously demonstrated using delivery passive RBC—hitchhiking. For example, lung targeting may be as much as 400% (injected dose/gram (ID/g) at 30 minutes) greater than delivery previously obtained by passive RBC-hitchhiking of targeted particles. In certain embodiments, administration of dual-targeted nanoparticles results in at least 2× greater delivery (e.g. to the lungs) than a passive RBC-hitchhiked particle targeted only to the lungs. In certain embodiments, administration of dual-targeted nanoparticles results in at least 2.5× increased delivery (e.g. to the lungs) compared to direct injection of free dual-targeted particles (i.e. not coupled to RBC ex vivo).

The biocompatible and/or non-toxic coupling of NP to the targeting moieties in these compositions allows ready transfer of the NP from the RBC to the end target, e.g., vascular endothelium, white blood cells, or other cells of the end organ. In certain embodiments, the target organ is the lungs, brain, or heart. In certain embodiments, the target is a blood clot (e.g. for treatment of ischemic stroke). In a method using dual-targeted NPs, i.e., in which the NP is associated with an antibody to RBC and an antibody to an antigen on a target cell, the delivery of a drug in the NP may be aided by covalent binding of the NPs to RBCs and then further targeting by the covalent binding of the antibody to the target antigen to the resulting target. Without wishing to be bound by theory, the method operates in the following manner: an RBC to which NPs are coupled ex vivo via a targeting moiety are administered to a patient and transfers the NPs to the surface of a vascular vessel, e.g., a capillary, due to a second targeting moiety specific for an endothelial antigen. In certain embodiments, the NPs are thereafter taken up by the target cell and the RBC is released. In certain embodiments, the NPs are nanogels carrying one or more selected drugs for delivery to the vessel or target organ.

In certain embodiments, the selected NPs can be optionally coated with a protein that does not induce an immunological reaction to the nanoparticle in a mammalian subject. By “mammalian subject” is meant primarily a human, but also domestic animals, e.g., dogs, cats; and livestock, such as cattle, pigs, etc.; common laboratory mammals, such as primates, rabbits, and rodents; and pest or wild animals, such as deer, rodents, rabbits, squirrels, etc. The selected coated or non-coated NP is then loaded with a suitable drug or multiple drugs generally by incubation at about 37° C. in a buffer. Desirable buffers are those that are RBC-compatible, such as phosphate buffered saline or the like. Other methods for drug loading in drug carriers include osmotic loading of a variety of small molecule drugs in protein, nanogel, or nanoparticle carriers either before or after linking the NPs and RBC. Drug loading and release from nanogels on RBCs may be modified by using crosslinkers incorporated in the nanogel to prolong or enhance encapsulation of loaded drugs and performing the crosslinking after drug loading. Crosslinkers can include responsive moieties (e.g. enzyme-cleavable crosslinkers that allow stimulated drug release in response to protease activity). In another embodiment, the drug is kept in the solution or in the wash buffer during all drug loading steps (except the last resuspension).

Also provided herein are methods for generating dual-targeted nanoparticle compositions. For example, dual-targeted nanoparticles having a first targeting moiety and a second targeting moiety, wherein the first targeting moiety is a red blood cell(RBC)-targeting moiety, is contacted with RBC, resulting in the dual-targeted nanoparticles being bound to the RBC. In certain embodiments, the NPs are bound to RBCs via a second targeting moiety prior to delivery in vivo and following drug loading. In certain embodiments, the RBCs are present in a sample of enriched or isolated (i.e., separated from one or more other components of whole blood) RBCs. In one embodiment, RBCs are in a sample obtained from a mammalian subject (e.g., an autologous donor or the subject to be treated) and bound to NPs before or after leukoreduction. In one embodiment, the RBCs are present in a whole blood sample isolated from the donor (e.g., an autologous donor or the subject to be treated). In another embodiment, the RBCs are isolated or enriched from a sample obtained from the donor (e.g., an autologous donor or the subject to be treated). In another embodiment, no leukoreduction is performed. In one embodiment, the source of the RBCs is a typed and crossmatched unit of peripheral RBCs. In another embodiment, the source of the RBCs is a universal donor (O-neg). In another embodiment, the source of the RBCs is the subject to which the dual-targeted nanoparticle composition is to be administered.

In yet another embodiment, a composition comprising dual-targeted nanoparticles is administered to a subject and the nanoparticles bind RBCs in vivo via a first targeting moiety.

Similarly, the methods may employ other adjunctive steps or devices to prepare the compositions described herein including microfluidic devices to put RBCs and NPs into small volume to increase adsorption efficiency or storage of NPs in bags that are RBC compatible, or use of bags that do not adsorb NPs, use of wide bore infusion tubing or tubing that doesn't adsorb nanoparticles, short-tubing to avoid adsorption to tubes, or use of transfer pipettes (large bore) during wash steps.

In certain embodiments, where the nanoparticles are hydrogels, the preparative method can involve retaining NPs in a solution containing a high concentration of a drug and centrifuging the NPs and resuspending same in a solution with a low concentration of said drug within 60 minutes before in vivo delivery. In another method, the binding step can occur by adding nanoparticles to a unit of red blood cells without excipients. In still another preparative method step, the binding can involve extracting blood from a mammalian subject and agitating a mixture of RBCs and loaded nanoparticles ex vivo within 60 minutes before in vivo delivery. In still a further modification, the nanoparticles are present in a syringe and binding of the NPs to RBCs via a first targeting moiety occurs when a mammalian subject's blood is withdrawn into the syringe. In another embodiment, if a small volume of RBCs is used, the container may be pre-coated with RBCs before NPs are introduced.

In another aspect, the compositions describe herein comprising dual-targeted NPs are useful in therapeutic treatment of disease, diagnosis of disease, or prophylactic treatments to prevent disease, depending upon the identities of the drugs with which the NPs are loaded.

By “disease” as used herein is meant, without any limitation, any disease in which small arterioles, capillaries, venules and/or the endothelial barrier plays an important role. Without limitation, such diseases and disorders include those involving the lung, including ARDS, IPF (idiopathic pulmonary fibrosis), pulmonary arterial hypertension, post-pulmonary embolism to prevent reactive vasoconstriction, pulmonary capilliaritis syndromes, such as the vasculitidies of granulomatosis and polyangitis (GPA) and Goodpasture's syndrome, among others. Still other diseases suitable for such treatment include those involving the heart, such as heart attack, ischemia-reperfusion injury, stroke or other diseases involving the heart; diabetic retinopathy or macular degeneration and other disorders involving the eye; hyperthyroidism or hypothyroidism and other diseases involving the thyroid; autoimmune hepatitis, alcoholic hepatitis, NAFLD/NASH and other diseases involving the liver; pancreatitis or other diseases of the pancreas; immunological disorders or other diseases of the spleen; inflammatory bowel disease and other diseases of the intestines; benign prostatic hypertrophy (BPH), prostate cancer, and other disease of the prostate; disorders of the brain and cancers of any organ or tissue. Still other diseases or conditions suitable for treatment, prophylaxis or diagnosis with compositions described herein include ischemic stroke, prevention of ischemia reperfusion injury (IRI), prevention of post-myocardial infarction, also for prevention of IRI, prevention or treatment of PAD (peripheral artery disease, especially the legs), prevention or treatment of cancer, especially head/neck cancer after subarachnoid hemorrhage, and encephalitis and meningitis. In still another embodiment, the disease or disorder may be a need for a transplanted organ, and the transplanted organ itself may be treated ex vivo with compositions described herein, e.g., to prevent IRI. This includes all solid organ transplants.

As used herein, the term “treatment,” and variations thereof such as “treat” or “treating,” refers to clinical intervention in an attempt to alter the natural course of the individual being treated, and can be performed either for prophylaxis or during the course of clinical pathology. Desirable effects of treatment include, but are not limited to, preventing or reducing the occurrence or recurrence of disease, alleviation of symptoms, diminishment of any direct or indirect pathological consequences of the disease, decreasing the rate of disease progression, amelioration or palliation of the disease state, and remission or improved prognosis. In some embodiments, effectors described herein are used to delay development of a disease or to slow the progression of a disease. In certain embodiments, the drug is released or transferred from the nanoparticles within 5, 10, 15, 20, 25, or 30 minutes of administration in vivo to a target organ. In certain embodiment, the composition contains a single drug or multiple drugs. In another embodiment, multiple drugs are administered simultaneously in the same or multiple RBC-coupled NP compositions. In certain embodiments, at least 50%, at least 60%, at least 70%, at least 80%, or at least 90% of the administered dose of a drug is delivered a target organ. In a certain embodiment, the target organ is the lung and at least 70% of the injected dose of the drug is delivered. In a further embodiment, where the target organ is the lung, delivery of dual-targeted nanoparticles results in enhanced delivery to the lung, reduced reticuloendothelial system (RES) clearance, plasma-half life and/or reduced toxicity or off target effects as compared to drug delivery using non-targeted NPs, single-targeted NPs, or RBCs with physisorbed NP or passive RBC hitchhiking.

By “administering” or “route of administration” is meant delivery of composition described herein, with or without a pharmaceutical carrier or excipient, to the subject. Routes of administration may be combined, if desired. In certain embodiments, the method includes intravenous delivery of a composition described herein. In certain the method includes intraarterial delivery of a composition described herein.

In certain embodiments, administration of the compositions described herein can be intravenous (iv) for delivery of the drug to the lungs. For example, where the disease is ARDS, pneumonia, interstitial lung disease, idiopathic pulmonary fibrosis, post-pulmonary embolism, pulmonary capilliaritis syndrome, emphysema, or a viral infection (such as SARS, influenza, or COVID). the compositions may be administered intravenously. In certain embodiments, the methods employ injecting iv the compositions described herein carrying one or more of albuterol, dexamethasone, and palifermin for the treatment of ARDS. In certain embodiments, where the disease involves any other selected mammalian organ, the composition is administered in vivo intra-arterially immediately upstream of the organ for delivery of effective doses of the drug. Additionally, for treatment of disease involving a selected mammalian organ designated for transplantation (other than the lung), the composition is administered ex vivo via feeding arterial opening into the organ prior to reperfusion and transplantation. In certain embodiments, the composition is administered via an arterial conduit of a selected organ using an arterial catheter. In any intra-arterial administration, the composition can be administered via an intra-arterial catheter. Such organs include, without limitation, the heart, brain, eyes, thyroid, kidney, liver, pancreas, spleen, intestines, or prostate.

Certain adjunctive steps for administering the compositions described herein include rotating or agitating or diffusing the RBC-coupled NPs before administering to prevent settling and aggregation of RBCs. In another embodiment, the method may employ a syringe that removes the RBCs from a patient's own blood and then reinjects after RBC-NP coupling. The syringe may be pre-loaded with anti-coagulant (that reverses upon re-injection to patient; e.g. drug marketed as Eloquis®), or a leukoreduction filter. An intra-arterial catheter may be employed that uses ultrasound to scan for the presence of arterial plaque prior to intracarotid injection, preventing embolization of atheromatous plaques. Similarly, a device to allow intracarotid (or other arterial injection) with simultaneous closure of the injection hole can be employed in these methods.

In still a further aspect, the compositions and methods can also be employed for imaging, such as to map capillary structure or pathology, wherein the drug is an imaging reagent. In one embodiment, a method of imaging a mammalian organ comprises injecting intravenously or intraarterially in vivo or ex vivo a composition comprising dual-targeted nanoparticles described herein, wherein the nanoparticles contain an imaging drug, and wherein the nanoparticles are transferred from the dual-targeted nanoparticles to the vascular bed of a target organ of the subject and release the imaging drug to the selected target organ or vascular tissue.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

Methods and agents well known in the art for making formulations are described, for example, in “Remington's Pharmaceutical Sciences,” Mack Publishing Company, Easton, Pa. Formulations may, for example, contain excipients, carriers, stabilizers, or diluents such as sterile water, saline, polyalkylene glycols such as polyethylene glycol, oils of vegetable origin, or hydrogenated napthalenes, preservatives (such as octadecyldimethylbenzyl, ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, and m-cresol), low molecular weight polypeptides, proteins such as serum albumin, gelatin, or immunoglobulins, hydrophilic polymers such as polyvinylpyrrolidone, amino acids such as glycine, glutamine, asparagine, histidine, arginine, and lysine, monosaccharides, disaccharides, and other carbohydrates including glucose, mannose, and dextrins, chelating agents such as EDTA, sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

All scientific and technical terms used herein have their known and normal meaning to a person of skill in the fields of biology, biotechnology and molecular biology and by reference to published texts, which provide one skilled in the art with a general guide to many of the terms used in the present application. However, for clarity, certain terms are defined as provided herein.

The terms “a” or “an” refers to one or more, for example, “an assay” is understood to represent one or more assays. As such, the terms “a” (or “an”), “one or more,” and “at least one” are used interchangeably herein.

The term “about” as used herein when referring to a measurable value such as an amount, a temporal duration, and the like, is meant to encompass variations of up to ±10% from the specified value; as such variations are appropriate to perform the disclosed methods. Unless otherwise indicated, all numbers expressing quantities of ingredients, properties such as molecular weight, reaction conditions, and so forth used in the specification and claims are to be understood as being modified in all instances by the term “about.”

Various embodiments in the specification are presented using “comprising” language, which is inclusive of other components or method steps. When “comprising” is used, it is to be understood that related embodiments include descriptions using the “consisting of” terminology, which excludes other components or method steps, and “consisting essentially of” terminology, which excludes any components or method steps that substantially change the nature of the embodiment or invention.

### EXAMPLES

The following examples disclose specific embodiments of preparation of compositions of this invention, their characteristics, and methods of use thereof. These examples should be construed to encompass any and all variations that become evident as a result of the teachings provided herein.

### Example 1: Methods and Materials

### Synthesis and Characterization of Dual-Targeted Liposomes

### Particle Synthesis

Azide functionalized PEG liposomes were prepared as described previously. ³⁷ Briefly, lipids DPPC (1,2-dipalmitoyl-sn-glycero-3-phosphocholine), cholesterol, and azide PEG₂₀₀₀ DSPE (1,2-distearoyl-sn-glycero-3-phosphoethanolamine-N-[azido(polyethylene glycol)-2000]) (Avanti Polar Lipids, Alabaster, Ala.) were combined with the phospholipid to cholesterol ratio at 3:1. Liposomes requiring ¹¹¹In radiolabeling include 0.2 mol % DTPA-PE (1,2-distearoyl-sn-glycero-3-phosphoethanolamine-N-diethylenetriaminepentaacetic acid), and those requiring fluorescence include 0.5 mol % Top FL-PC (1-palmitoyl-2-(dipyrrometheneboron difluoride) undecanoyl-sn-glycero-3-phosphocholine) or [TopFl-PE, Rhodamine-PE. Lipid solutions were subjected to a constant stream of nitrogen gas until visibly dried, then lyophilized for 1-2 h to remove residual solvent. Dried lipid films were rehydrated with buffer, either sterile PBS or 0.3N metal free citrate at pH 4. This lipid solution underwent 3 cycles of freeze thaw between liquid N₂ and a 50° C. water bath, followed by 10× extrusion cycles through 200 nm polycarbonate filters using an Avanti Mini Extruder (Avanti Polar Lipids). At each stage of particle synthesis and modification, particle size, distribution, and polydispersity index (PDI) were taken at 1:125 dilution in PBS using a Zetasizer Nano ZSP. Particle concentration in #/ml was measured using a NanoSight NS300 at a dilution in ultrapure DI water of ˜10⁴. (Both instruments by Malvern Panalytical, Malvern UK.)

### Modification of Targeting Monoclonal Antibodies

As described previously⁵¹, highly stable and homogeneous immunoliposomes were synthesized using copper free click chemistry methods. All monoclonal antibodies and control IgG were modified with dibenzylcyclooctyne-PEG4-NHS ester (Jena Bioscience; Thuringia, Germany). The proteins, buffered in PBS and adjusted to pH 8.3 with 1 M NaHCO3 buffer, were reacted for 1 h at room temperature (RT) at a ratio of 1:5 antibody/NHS ester PEG₄ DBCO. Post reaction, the mixture was buffer exchanged with an Amicon 10 k MWCO centrifugal filter (MilliporeSigma, Burlington Mass.) to remove unreacted NHS ester PEG₄ DBCO by 30 vol washes. The efficiency of DBCO-IgG reaction was determined optically, with absorbance at 280 nm indicating IgG concentration and absorbance at 309 nm indicating DBCO concentration. Spectral overlap of DBCO and IgG absorbance was noted by correcting absorbance at 280 nm. Molar IgG concentration was determined using Beer's Law calculation, with an IgG extinction coefficient of 204,000 L mol⁻¹cm⁻¹ at 280 nm. Likewise, the molar DBCO concentration was determined using the DBCO extinction coefficient at 309 nm, 12,000 L mol⁻¹ cm⁻¹. The number of DBCO per IgG was determined as the ratio. All Ab-DBCO used in these studies had between 2-5 DBCO/Ab.

Monoclonal antibodies modified included those against endothelial targets intracellular adhesion molecule (ICAM-1) and platelet-endothelial cell adhesion molecule (PECAM-1) for both mouse (YN1 and Mec13, respectively) and human (R6.5 and Ab62) and those against RBC target GPA both mouse (Ter119) and human (CD235), and Rh in human (Bric69). Whole molecule rat IgG was included for controls, and as a non-immune vehicle for ¹²⁵I to quantify particle localization. Radiolabeling of IgG-DBCO with Na-¹²⁵I was done using the iodogen method as already described.⁵² For quantification of conjugation individual antibodies in dual preparations, Ab-PEG₄-DBCO were further modified with either NHS-Alexafluor 488 or 594 as directed by the manufacturer (ThermoFisher, US), and purified using Amicon filters as described.

### Radiolabeling DT Liposomes

Liposomes were isotope traced either by inclusion of ¹²⁵I-IgG/DBCO on the surface of the particle at no more than 10% of total antibody coating or by surface chelation of ¹¹¹In to DTPA-PE on the particle surface as already described.³⁷ IgG-DBCO underwent radio-iodination with Na-¹²⁵I using the iodogen method. Surface chelation of ¹¹¹In was done using metal free conditions to reduce reaction inefficiencies due to metal contamination. ¹¹¹In Cl3 (Nuclear Diagnostic Products, Cherry Hill, N.J.) was diluted in citrate buffer and added to preformed azide 0.2% DTPA liposomes, hydrated with metal-free pH 4 citrate buffer, and reacted for 1 h at 37° C. The reaction mixture was quenched with 50 mM DTPA to 1 mM final concentration to chelate unincorporated ¹¹¹In. The radiochemical purity and yield quantified using thin film chromatography (TLC) with mobile phase EDTA 10 μM gamma counting of the aluminum silica strips (Sigma Chemical, St Louis Mo.). For biodistributions liposomes were labeled at 50-100 μCi/μmol. Liposome samples were buffer exchanged with sterile PBS using Amicon centrifugal filters, followed by targeting ligand conjugation.

### Ligand Conjugation and Characterization of Dual-Targeted Immunoliposomes

Antibodies were conjugated to liposomes using copper-free click chemistry as previously described.³⁷ DBCO-functionalized monoclonal antibodies described earlier were incubated with azide-bearing liposomes from 4 h to overnight at 37° C. with rotation. Post incubation mixtures were purified using size exclusion chromatography using Sepharose 4B-Cl (GE Healthcare, Pittsburgh Pa.) packed in a 20 mL Biorad polyprep column taking 1.0 mL fractions for 25 mL, quantification of binding was done via tracing ligand fluorescence. Dual antibody formulations were characterized individually using different fluorophores conjugated to the proteins directly as described, e.g. Alexafluor 488 for YN1 and Alexafluor 594 for Ter119, with fractions read on a plate reader (Spectramax M2; Molecular Devices, San Jose, Calif.) or radioactivity (fractions measured on a gamma counter). Efficiency of conjugation reaction is quantitatively defined as the ratio of the area under the curve of the ligand signal in the liposome peak (4.0-6 mL) over the integration of the entire 25 mL elution plotted by signal over elution volume (FIG. 1E, FIG. 1F).

### Red Blood Cell (RBC) Preparation and Liposome Loading for In Vivo, In Vitro, and Ex Vivo Studies

### RBC Isolation, Purification, and ⁵¹Cr Labeling

Murine RBC were obtained from male C57BL/6 mice (The Jackson Laboratory, Bar Harbor, Me.) by inferior vena cava puncture after anesthesia with ketamine/xylazine (100/10 mg/kg). Human RBC were obtained by sterile venipuncture from healthy adult donors. For ex vivo lung perfusion (EVLP) experiments, donor RBC blood type was matched to the blood type for donor lung tissue. Murine and human RBC were treated and washed identically after blood draw. To prevent coagulation, syringes and collection tubes were pre-treated with ethylenediaminetetraacetic acid (EDTA, Sigma Aldrich, St Louis, Mo.), in DPBS (Corning, Manassas, Va.). RBC were purified from WBC, platelets, and serum by centrifugation and washing 2× with DPBS. RBC were either used immediately or resuspended in 5 mMol glucose in DPBS (Sigma Aldrich, St Louis, Mo.) for storage up to 24 h at 4° C.

When RBC tracing or labeling was required, RBC were resuspended at 10% hematocrit (hct) in 5 mM glucose and incubated with chromium-51 radionuclide (⁵¹Cr, sodium chromate in normal saline, Perkin Elmer Life & Analytical Sciences) for up to 12 h at 4 C. RBC were washed 2× with DPBS to remove free ⁵¹Cr and either used immediately or stored as previously described.

### Liposome-RBC Loading

RBC were isolated and purified as described. Loading was found to have the highest efficiency when performed at higher RBC concentration (hematocrit) and given at least 90 minutes for binding (supplemental Fi) so liposomes were highly concentrated to maintain a RBC hematocrit of approximately 50% after mixing. Liposome/RBC mixtures were incubated at 4° C., rotating, for 90 minutes in Axygen maximum recovery microtubes (Corning, Mexico). After incubation with liposomes, RBC were washed 2× with DPBS to remove unbound liposomes then the washes and remaining pellet were measured for radioactivity using a Wallac 1470 Wizard gamma counter (Perkin Elmer Life and Analytical Sciences-Wallac Oy, Turku, Finland). Liposome loading efficiency was calculated from radioactivity remaining in the RBC pellet after washing divided by radioactivity in the pellet plus washes.

### Liposome-RBC Binding, Immunoreactivity, Agglutination, and Flow Cytometry

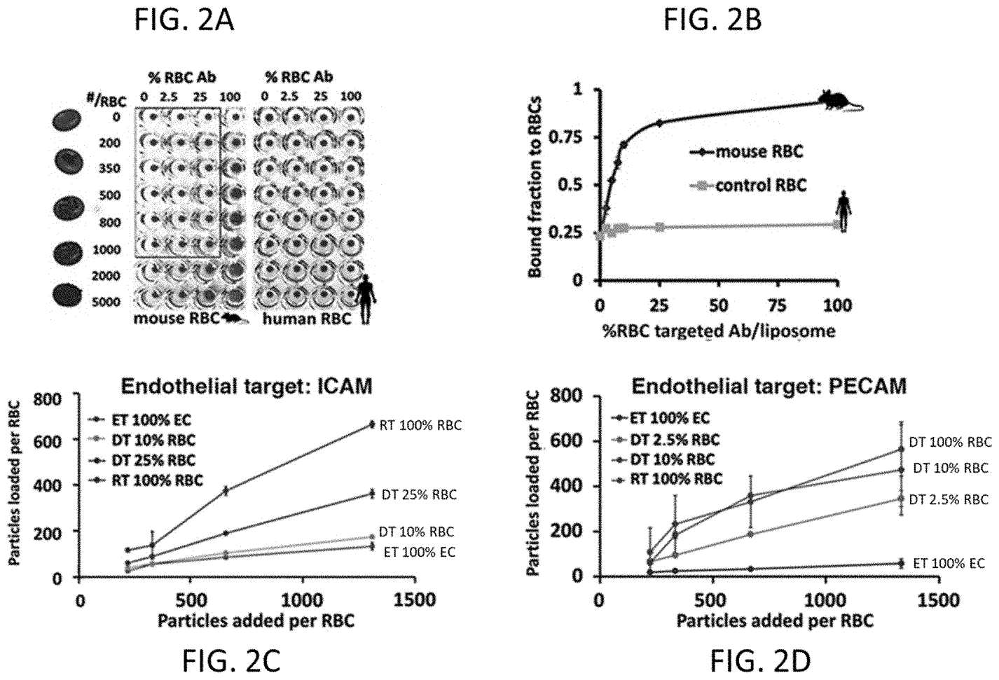

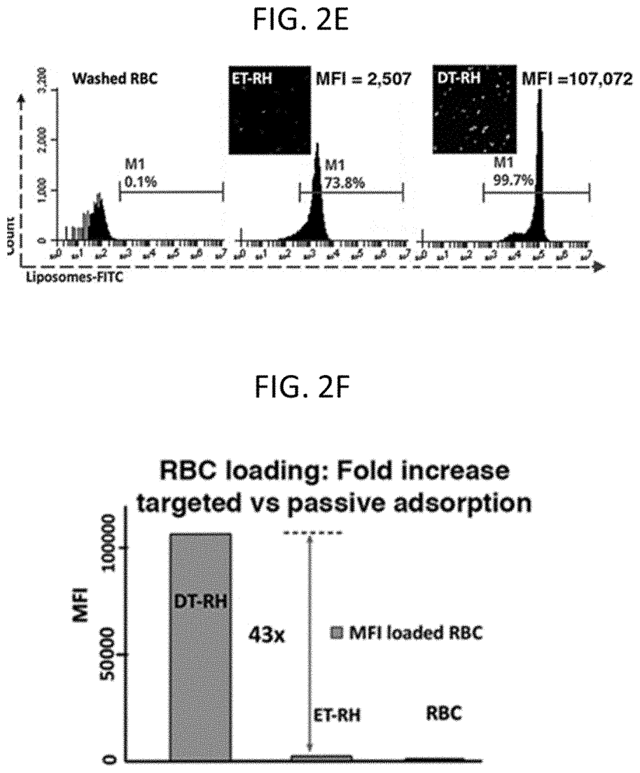

A standard agglutination assay was performed as is done clinically.⁵³ The assay was performed at 2% hct: 20 uL of pRBC with a varied number of liposomes (FIG. 2A) were resuspended in 200 uL DPBS in a round-bottom 96 well plate (Thermo Fisher Scientific, Denmark). RBC were allowed to settle for 2 hours then observed and photographed for agglutination. Agglutination is assessed visually as the absence of a clean-bordered well-demarcated pellet. Immunoreactivity and binding assays were conducted similarly to loading, with a varied number of liposomes added per individual RBC. Flow cytometry of loaded RBC was performed on an Accuri C6 (BD Biosciences) and analysis done using FCS express.

### Naming Conventions for Targeted Liposomes

Naming conventions used hereafter in Example 1 and Example 2 are diagrammed in FIG. 1D and explained in the table below. Endothelial targeted (ET) refers to liposomes that are single-targeted to CAM epitopes only. CAMs included here were either Platelet Endothelial Cell Adhesion Molecule (PECAM) or Intercellular Adhesion Molecule 1 (ICAM). RBC targeted (RT) refers to liposomes that are single-targeted to RBC only. Dual-targeted (DT) refers to liposomes that are targeted to both a CAM epitope on EC and a surface epitope on RBC. Liposomes that were injected without first being adsorbed onto RBCs are simply called free liposomes.

### Naming Conventions for Targeted Liposomes

| Abbreviation | Description |
|---|---|
| DT | Dual-targeted liposome. Conjugated to 2 types of |
| liposome | antibodies, one binding RBCs and one binding endothelial |
| | cells. |
| ET | Endothelial Targeted liposome. Contains only antibodies |
| liposome | binding endothelial cells. |
| RT | RBC Targeted liposome. Contains only antibodies binding |
| liposome | to RBCs. |
| RH | RBC-hitchhiking. Passive RH refers to an RBC-liposome |
| | complex where liposomes were passively adsorbed onto |
| | RBC via an unknown mechanism prior to injection. |
| Free | A liposome of any formulation that is injected in vivo |
| liposome | without being loaded on an RBC first |
| DTRH | DT liposomes that were first loaded ex vivo onto RBCs |
| | before IV injection. |
| ET-RH | ET liposomes that were first loaded ex vivo onto RBCs |
| | before IV injection. |
| RT-RH | RT liposomes that were first loaded ex vivo onto RBCs |
| | before IV injection. |

### Animal Studies: Biodistribution, Flow Cytometry, Microscopy

### Biodistribution and Pharmacokinetic Studies

Naïve C57BL/6 male mice (The Jackson Laboratory, Bar Harbor, Me.) anesthetized with ketamine/xylazine (100/10 mg/kg) were injected intravascularly with 1 μmol (0.75 mg) total radioimmunoliposome dual conjugated with targeting ligand against ICAM or PECAM antibody, Ter119 against GPA on the RBC, and ¹²⁵I-IgG). Animals were euthanized at designated times after injections; the organs of interest harvested, rinsed with saline, blotted dry, and weighed. Blood samples (˜200 ul) were spun down at 500 rcf in a microcentrifuge tube with RBCs separated from plasma. Radioactivity in organs and separated blood components were measured with a Wallac 1470 Wizard gamma counter (PerkinElmer Life and Analytical Sciences-Wallac Oy, Turku, Finland). The gamma data of the ¹²⁵I and ⁵¹Cr (or ¹¹¹In) measurements and organ weights were used to calculate the tissue biodistribution injected dose per gram. The total injected dose was measured prior to injections, corrected for tube and syringe residuals, and verified to be ≥75% of the sum of the individual measures.

### Flow Cytometry Analysis of Dual-Targeted Liposomes and Single-Cell Preparation of Lung Homogenate

Following intravenous administration of dual-targeted liposomes that were either injected freely (direct injection) or loaded ex vivo onto RBC, lung tissue was prepared for flow cytometry to determine which cell types liposomes were delivered to. At 30 minutes, a tracheostomy and cannulation were performed then animals were sacrificed. The right ventricle was cannulated and perfused with cold PBS at 20 cm H₂O to flush RBC from the pulmonary capillary bed. Lungs were re-inflated with 0.8 mL digestive enzyme solution (collagenase type 1 (Life tech), dispase (Collaborative), DNase1 (Roche) with PBS) and removed from the chest cavity. Harvested lungs were prepared into single cell suspension first by manual chopping with addition of additional digestive enzyme. Samples were incubated in 37° C. water intermittently vortexed then mixed with fetal bovine serum (Sigma, Pa.). Homogenate was strained through a 100-micron filter, centrifuged, and resuspended in ACK lysing buffer (Gibco) to remove RBC, then strained through a 40-micron filter on ice, centrifuged, and resuspended in FACS buffer (1% FBS, 1 mM EDTA in PBS, reagents already specified). Cells were fixed then centrifuged and resuspended in FACS buffer for flow cytometry. This single cell suspension was stained for CD45 (Anti-mouse CD45-brilliant violet 421, BioLegend) and PECAM (Anti-Mouse-CD31-APC, Invitrogen, CA). Final resuspension in 2:2000 DAPI was used to exclude dead cells. Flow cytometry was performed on an LSR Fortessa (BD Biosciences) then gated for viability and singlets and analyzed with FlowJo software (FlowJo LLC).

### Microscopy Studies

For in vitro analysis of RBC binding with FITC-labeled TER119-coated liposomes RBC were incubated with the liposomes, washed by centrifugation, adsorbed on glass slides, washed and mounted. In in vivo studies animals were sacrificed; lungs were harvested, immersed in OCT, and frozen by liquid N₂. Frozen tissues were cut using Cryostat with 10-20 μm/slice. Samples were fixed with 4% paraformaldehyde for 10 min and permeabilized with 0.3% Triton X-100 for 15 min prior staining with antibodies. Leukocytes were stained with rabbit anti-mouse CD45 antibody (Abcam, #ab10558) followed by Alexa Fluor 647 labeled anti-rabbit IgG. Liposomes were stained with Alexa Fluor 594 goat anti-rat IgG (Invitrogen). Microscopy studies were performed on a confocal laser scanning microscope Leica TCS-SP8 (Leica, Germany) using HC PL APO CS2 63x/1.40 Oil objective and 488/552/638 lasers. Image analysis was performed using Volocity 6.3 Cellular Imaging & Analysis.

### Human Lung Studies

### Ex Vivo Lung Perfusion

Human lungs were obtained following organ harvest from transplant donors after lung tissue was deemed unsuitable for transplantation. The lungs were perfused and harvested by the organ procurement team and kept submerged in PBS at 4° C. until use in the lab, within 24 hours of harvest. The lungs were accepted for research if oxygenation, cause of death, and visual assessment was all consistent with normal lung function. We used a modified ex vivo lung perfusion (EVLP) protocol.⁵⁴ The airway was cannulated and inflated with low pressure oxygen; oxygen flow was continued at approximately 0.8 L/min to maintain gentle inflation. A subsegmental branch of the pulmonary artery was cannulated and perfused with Steen solution for 5 minutes at 20 cm H₂O. Green tissue dye was used to test for retrograde flow and identify efflux from the pulmonary vein. Using RBC labeled with ⁵¹Cr and DT liposome labeled with ¹²⁵I, a 3 mL DTRH sample was perfused by slow push into the arterial cannulation. This was chased with 3 mL of tissue dye to achieve bright staining of the perfused area of tissue. Finally, Steen solution was perfused for 10 mg at 20 cm H₂O. All efflux was collected from the pulmonary vein. The lung tissue was then dissected and areas perfused by green tissue dye were measured for retention of liposomes and RBC using ⁵¹Cr and ¹²⁵I signal measured by gamma counter.

### Example 2: Dual-Targeted Red Blood Cell Hitchhiking

Although the primary focus of targeted drug delivery is to concentrate drugs to a single cell type or organ, the majority of systemically administered drug nanocarriers never reach their target. Here, we combined a nanocarrier targeting approach of ligand (antibody) targeting with cell-based delivery to localize a majority of injected nanocarriers to their target. In dual-targeted RBC-hitchhiking (DTRH), liposomes are conjugated with two antibody types: one targeting red blood cells (RBCs) and a second targeting epitopes expressed in target vasculature. We demonstrated that DTRH localizes nanocarriers to the lungs at as high as 65% of the injected dose, >2.5 times that of comparable single antibody targeting. DTRH also improved delivery to specific cell populations (e.g., alveolar endothelial cells) within the target organ by >6.4-fold. Finally, we confirmed that DTRH enabled specific delivery in fresh ex vivo human lungs. Thus, DTRH shows great potential to improve organ- and cellular-level localization of therapeutics.

Design of Liposomes with Dual Avidity to RBC and Endothelial Cells (EC)

Dual-targeted (DT) liposomes (FIG. 1A) were designed to facilitate consistent, efficient, and transient binding to RBC and terminal binding to EC. FIG. 1B, left to right, shows a sequence of interactions between DT liposomes, RBC, and EC. First, DT liposomes bind to RBC ex vivo; after injection RBC-bound DT liposomes travel together as a complex in the bloodstream; next, when an RBC-DT liposome complex encounters the target microvasculature, engagement with the EC favors secondary binding to specific endothelial surface determinants; and finally, multivalent anchoring on EC causes DT liposomes to detach from carrier RBC, which are free to continue in circulation. In this manner, DT liposomes complete their transfer from circulating RBCs and on to the target EC, providing an unprecedented up to 65% of total injected dose to the lungs (FIG. 1C).

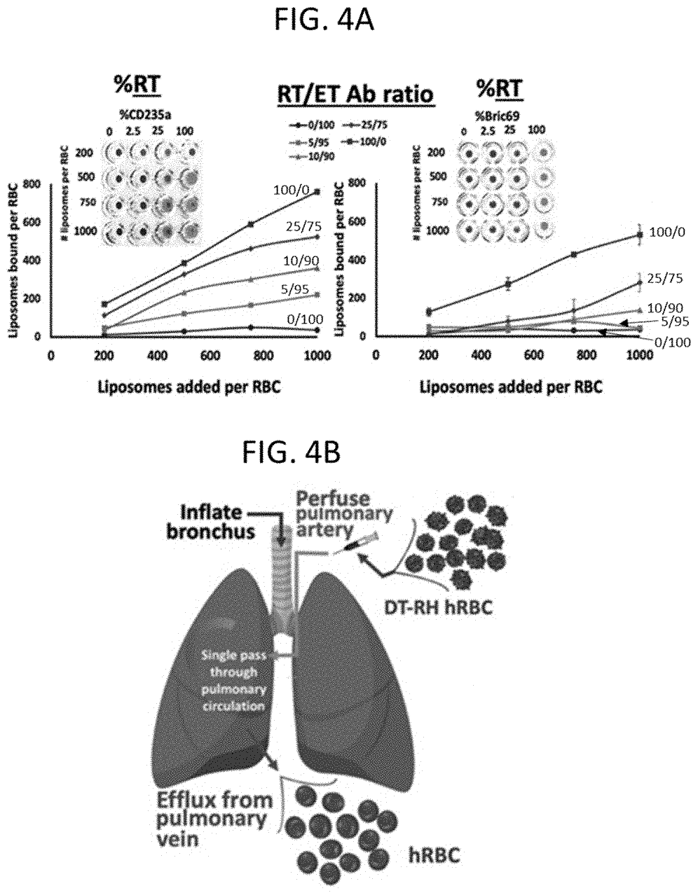

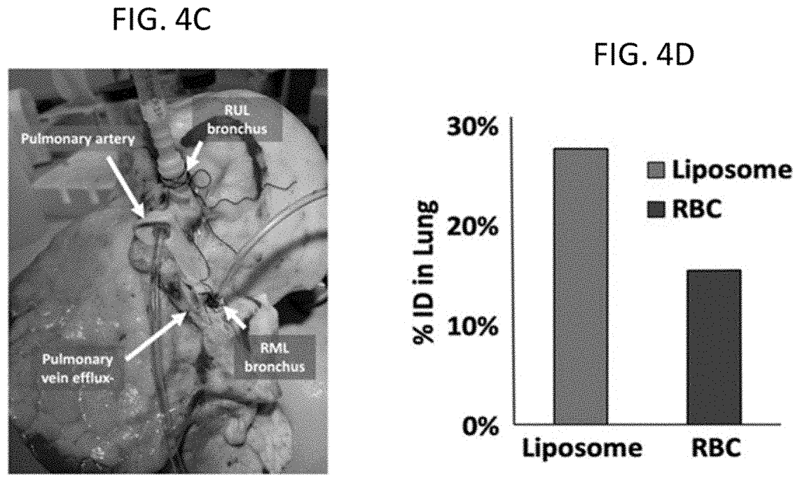

The components of DT liposomes are depicted in FIG. 1A. Tracing of liposomes was done using either ¹²⁵I-labeled rat IgG (yellow in the diagram) or ¹¹¹In-DTPA-conjugated to a membrane lipid. RBCs were traced by ⁵¹Cr labeling. Such DT liposomes were compared to liposomes targeted to only one cell population, EC or RBC (FIG. 1C, FIG. 1D; FIG. 4D). Endothelial targeted (ET) refers to liposomes that are single-targeted to EC using cell adhesion molecule (CAM) ligands. CAMs included here were either Platelet Endothelial Cell Adhesion Molecule (PECAM) or Intercellular Adhesion Molecule 1 (ICAM-1). RBC targeted (RT) refers to liposomes that are single-targeted to RBC using Glycophorin A (GPA) ligands. DT-liposomes are targeted to both GPA and CAM (ICAM or PECAM). These three target transmembrane glycoproteins are stably expressed at a level of ˜10⁶ copies per EC and RBC, respectively. Animal studies have established that conjugation with anti-GPA provides targeting of therapeutic proteins to RBC³⁵ and conjugation of diverse pharmacologic cargoes with anti-CAM provides efficacious targeting to the pulmonary vasculature.^{7,8,36-42}

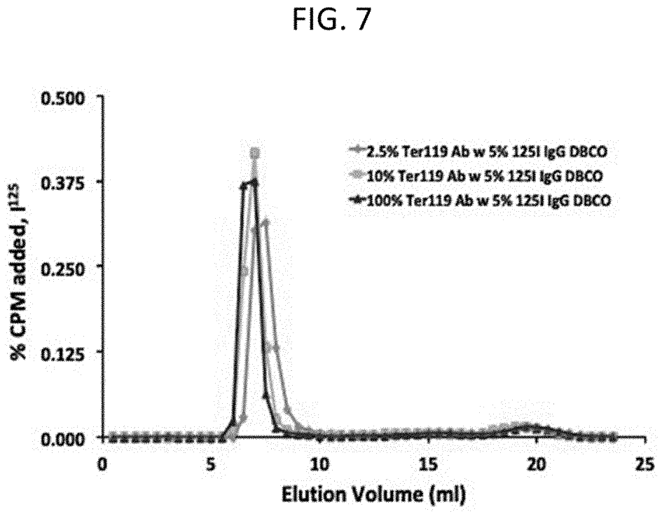

While DTRH is a fairly straightforward concept, its realization relies on balancing several challenging dualisms. For example, the surface of RBC contains abundant GPA. This is favorable for liposome loading and delivery, but unfavorable if multivalent anchoring occurs and prevents liposomes from transferring to EC. We varied the ratio between anti-GPA and anti-PECAM on the liposome surface, keeping a constant 200 monoclonal antibodies per liposome. FIG. 1D shows the hydrodynamic radius for each of the liposome variants, as measured both by dynamic light scattering (DLS) and nanoparticle tracking analysis (NTA). The addition of 200 antibodies per liposome uniformly added 15-20 nm compared to an unconjugated liposome. DT, RT, and EC liposomes all had a stable size of approximately 150 nm with no difference in size or particle distribution (PDI<0.2) among the formulations. FIG. 1E and FIG. 1F show that the efficiency of antibody conjugation to the liposome surface was >90% across all formulations studied, as measured by gel exclusion chromatography elution profiles. Area under the curve analysis from tracing fluorescently labeled ET or RT antibodies at titrated amounts demonstrates conjugation precision and consistency. Single DT liposome formulations contribute to the elution curves representing EC bound antibodies on liposomes in E and RT in F at each ratio using two different fluorescent tags, e.g. 97.5% EC antibody in FIG. 1E corresponds to 2.5% RT in FIG. 1F, both representing the same DT liposome sample. Insets for both FIG. 1E and FIG. 1F show the linear relationship across the titrated amounts of antibody of both types as derived from area under the curve analysis. Fluorescently labeled antibodies conjugated to liposomes elute consistently at between 6-8 ml, and free unbound fluorescent antibodies at 12-14 ml. Efficiencies are derived from the ratio of those two area peaks. Equivalent results were seen using radiolabeled antibodies for isotope tracing (FIG. 7). These methods used in conjunction with NTA facilitated consistent and precise command over dual antibody pairing ratios.

Dual-Targeted Liposome Interaction with RBC In Vitro

The DT liposome variants described in FIG. 1D were bound to the surface of murine RBC in vitro (called “RBC loading”) and evaluated for binding characteristics and their effect on RBC. High concentrations of anti-GPA antibodies against RBCs are known to damage the cells and cause RBC agglutination.³⁵ Thus, the bounds of both the percentage of anti-GPA antibody that can safely be added to a liposome's surface as well as the relative numbers of those coated liposomes that can be added per RBC need to be taken into consideration. All murine experiments used the antibody clone Ter119 against GPA. RBC agglutination resulting from contact with anti-GPA bound to liposomes is shown in FIG. 2A in the standard clinical “round bottom well assay.” RBC that do not agglutinate form a small circle in the well, while agglutinated RBCs form a broad mat. Agglutination was evaluated upon additions of EC-targeted (ET), DT, and RBC-targeted (RT) liposomes, with anti-GPA antibody coating 0, 2.5, 25 and 100% of the liposome surface respectively, right to left. The balance of antibody surface coating was taken by anti-ICAM antibody, YN1 clone. Total liposomes added per RBC ranged from 0 to 5000, reading top to bottom. ET liposomes and DT liposomes carrying 25% or less anti-GPA, but not RT liposomes, had no significant RBC agglutination at liposome: RBC ratios of less than 1000 liposomes per RBC, demonstrated by the box in FIG. 2A. Additionally, there was no effect of murine RBC-targeted liposomes on human RBCs at any condition tested, as seen on the entire right section of FIG. 2A, regardless of concentrations of either liposomes or density of RT antibody coating.

Continuing to define DT liposome formulations' interaction with RBC in vitro, the loading characteristics of DT liposomes onto RBC were determined by immunoreactivity and flow cytometry. DT liposomes isotope labeled with ¹²⁵I were tested against a vast excess (>1000×) of GPA binding sites. As the % RBC antibody (with anti-ICAM antibody filling in the remainder) reaches between 10-25%, the fraction of particles bound to the RBC approaches an asymptote (FIG. 2B). Liposome binding to human (control) RBCs remains nonspecific across all formulations. Thus, RT liposomes and DT liposomes bind specifically and proportionally to RBC based on the both density of anti-GPA antibody and the number of liposomes per RBC (FIG. 2A and FIG. 2B).

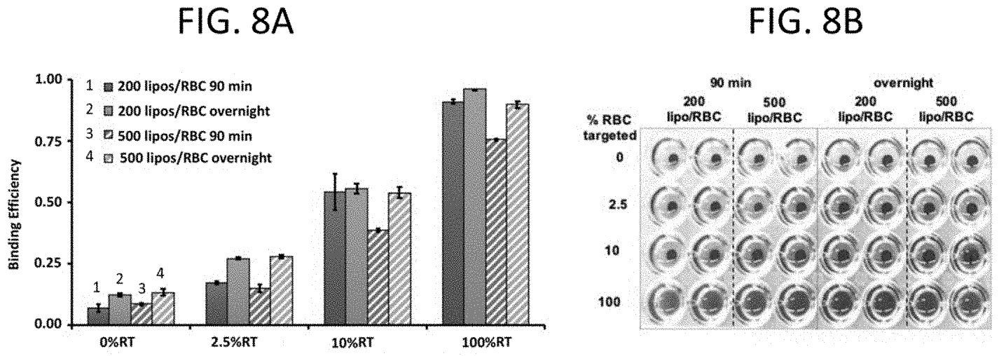

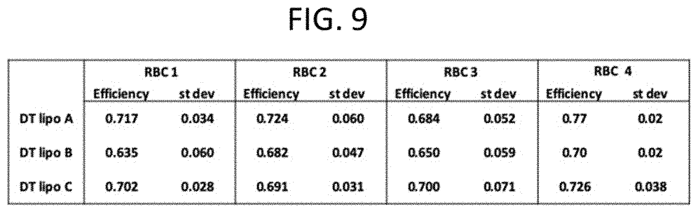

Having established the above parameters, two formulations of DT liposomes were chosen to quantify the effective ex vivo loading of liposomes onto RBCs for injection in vivo. Shown in FIG. 2C the two targets remain GPA (10 or 25% of liposome surface antibodies) and ICAM (90 or 75% of liposome surface antibodies, respectively) with comparison to ET liposomes 100% targeted to ICAM and RT liposomes 100% targeted to GPA. ET liposomes passively adsorbed onto RBCs, as found in prior studies on passive RH.¹⁸ DT liposomes bound with higher affinity than ET liposomes but lower affinity than RT liposomes. Across all four types of liposomes, RBC-binding was proportional to the fraction of surface antibodies targeting RBCs. Loading was predictable and linear with respect to the number of liposomes added per carrier RBC, without reaching saturated binding in the range used in this study. It is important to note that a distinction between nonspecific (ET liposome) binding and specific binding of DT liposomes occurred at 10% of liposome surface targeted to RBC. Efficiency of binding of liposomes to RBC and potential for harmful agglutination of those bound RBCs with respect to both time and bulk number of liposomes was established (FIG. 8A and FIG. 8B). Variability of binding efficiency as it related to individual animal source, or identical replicate DT liposome preparation and day/prepares hands was tested (FIG. 9) and found to not be statistically different either comparing source or liposome preparation. In comparison, FIG. 2D shows RBC binding when PECAM-targeting antibodies (clone Ab62) were substituted for ICAM-targeting antibodies on the surface of DT liposomes. Here, a distinction between the nonspecific binding of ET liposomes and DT liposomes is observed with only 2.5% of surface antibodies targeted to GPA. Additionally, increasing GPA targeting surface antibodies to 10% results in binding that is similar to that seen with RT liposomes. While the reasons for the difference in RBC loading with different anti-CAM antibodies are unclear, our results are validated by in vivo experiments with GPA/ICAM and GPA/PECAM DT liposomes.

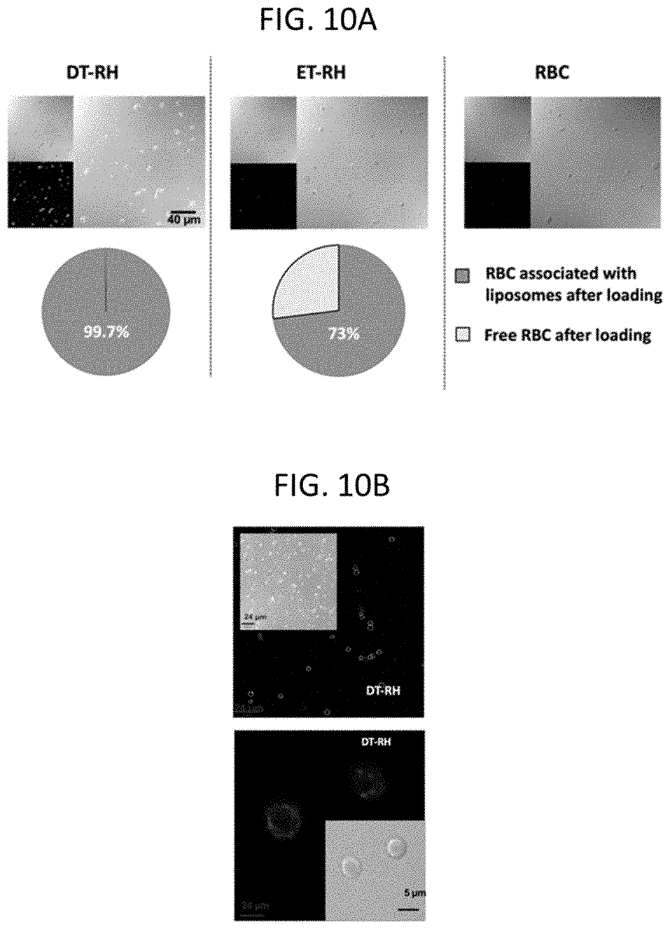

Lastly, the loading characteristics onto RBC were quantified using flow cytometry and visualized with confocal microscopy. Based on the results described above, DT liposomes were formulated with 200 antibodies on their surface; 10% anti-RBC and 90% anti-EC. To simulate untargeted RH, ET liposomes were composed 100% of anti-EC. After loading, DT liposomes coated 99.7% of the RBC population (FIG. 2E, right), compared to ET liposomes that bound only 73% of the RBC population (FIG. 2E, center). Additionally, the median fluorescence intensity (MFI) of RBCs bound by DTRH was 43× higher than that of ET-RH, indicating that more DT liposomes bound individual RBC. This was confirmed visually on a thin smear of liposome-loaded RBC (FIG. 2E, insets; FIG. 10A-FIG. 10B, RBC images). Therefore, DT liposomes yield more effective and homogeneous loading compared to ET liposomes (untargeted RH).

Dual-Targeted Liposomes are Precisely Formulated to Maintain Organ-Specificity and Improve Targeting Efficacy without RBC Retention in the Lungs

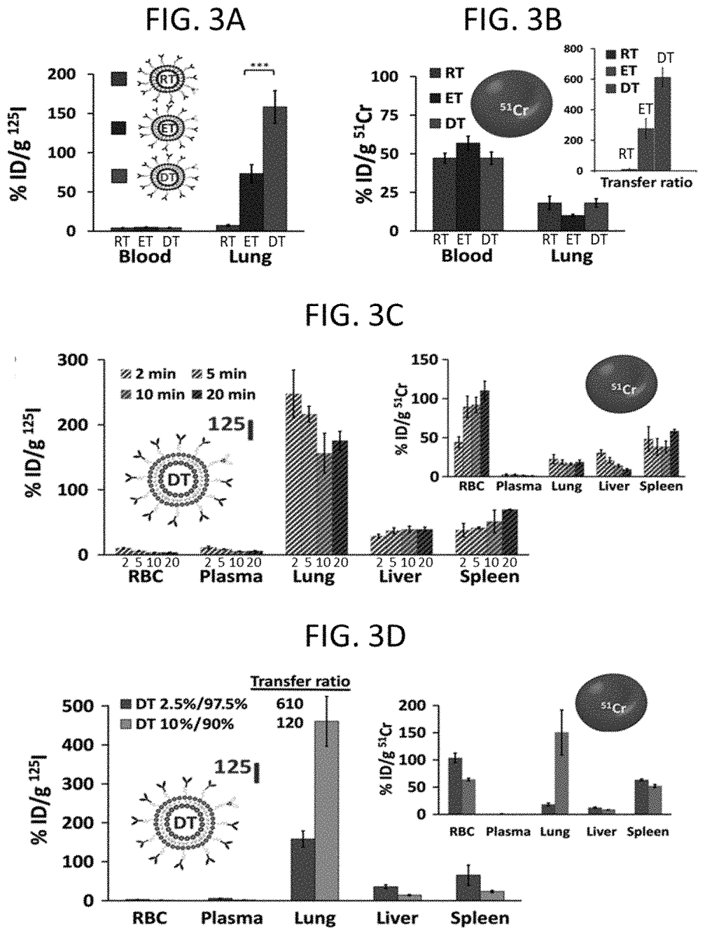

The in vivo behavior of ¹²⁵I-labeled liposomes loaded onto ⁵¹Cr-labeled RBC was characterized by comparing the following formulations, based on in vitro data (FIG. 2D): RT liposomes (2.5%:97.5% for a total of 5 RBC targeting molecules and 195 filler IgG molecules), ET liposomes (0%:100% for a total of 200 EC targeting molecules), and DT liposomes (2.5%:97.5% for a total of five RBC targeting molecules and 195 EC targeting molecules). The RBC surface was targeted with anti-GPA, and EC were targeted with anti-PECAM. Liposomes were loaded onto RBC and these liposome-RBC complexes were injected IV. Mice were sacrificed after 30 minutes and isotopes individually tracing both the liposomes and carrier RBC were measured in all major organs and the blood compartment (FIG. 3A and FIG. 3B). Initial organs of interest include the blood and lung compartments. ¹²⁵I deposition in the lung reveals that ET, but not RT liposomes accumulate in the pulmonary vasculature. This was expected based on ET liposome targeting and previous results showing PECAM specificity. More importantly, in spite of this high efficacy, the pulmonary uptake of DT liposomes doubled that of ET liposomes. No liposome formulations remained in the blood. Tracking ⁵¹Cr reveals that within a half hour, the RBC carriers for all three liposome formulations was nearly uniformly low in the lungs and high in the blood, indicating that each liposome formulation separated from their carrier RBC, which continued in circulation.

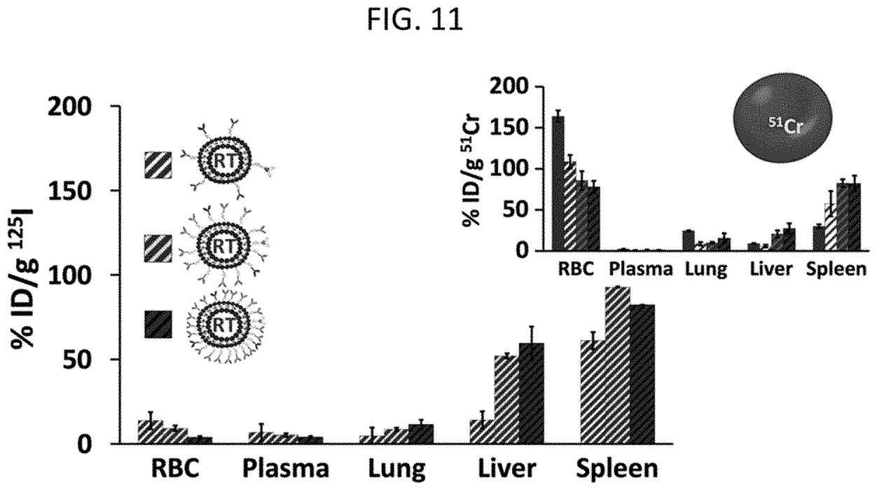

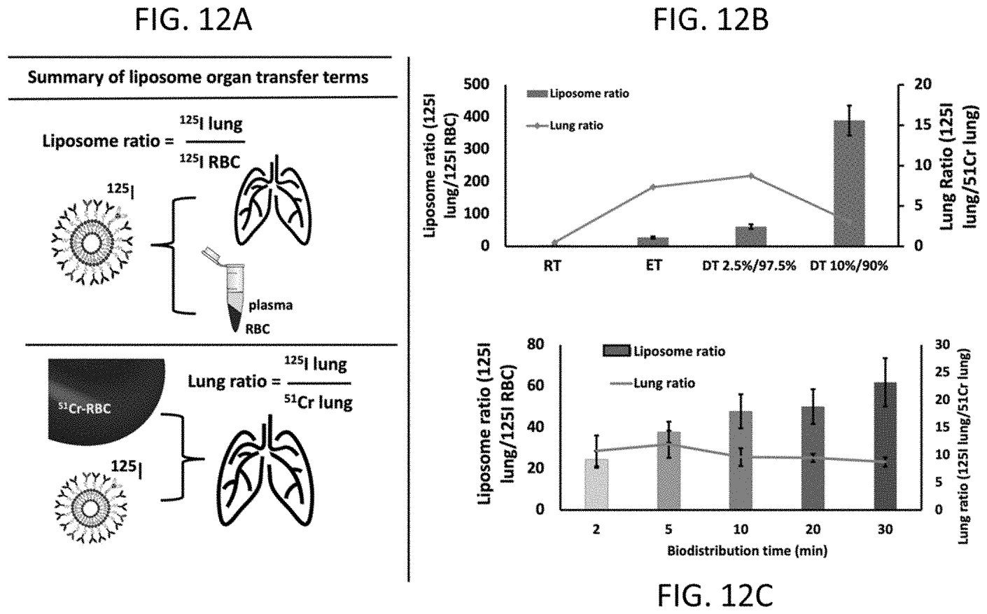

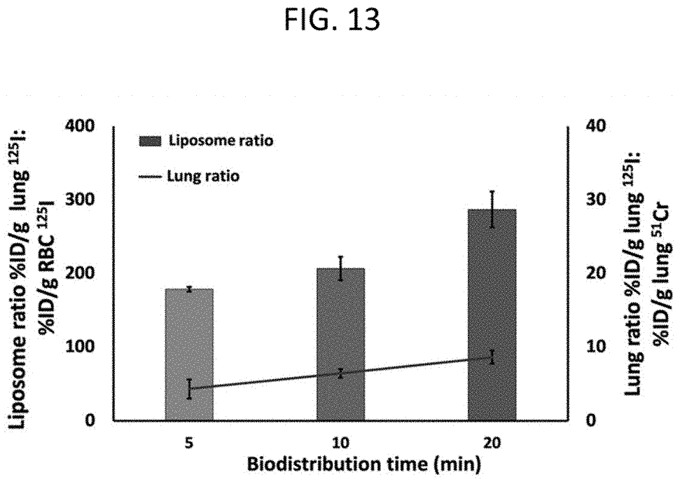

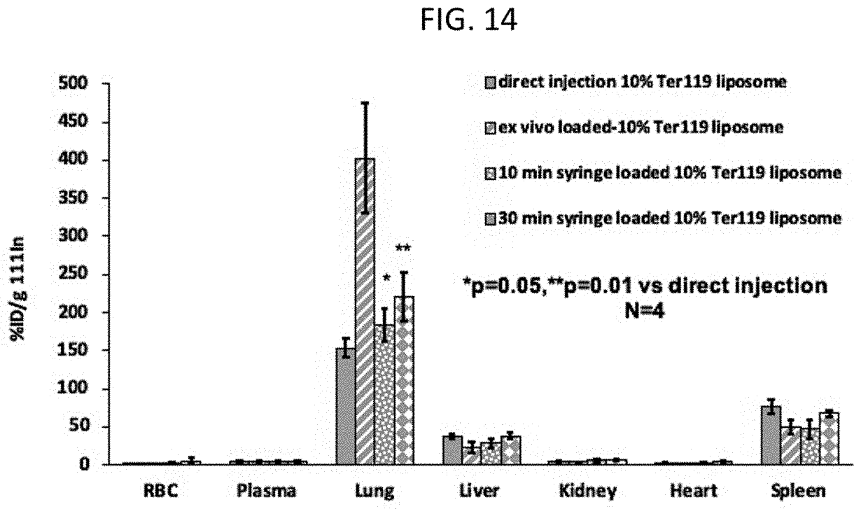

At first glance, this result may appear to contradict the following logic. Pulmonary vascular targeting by ET liposomes is known to be mediated by endothelial avidity provided by anti-PECAM; however, DT liposomes actually have a portion of their anti-PECAM replaced by anti-GPA. Therefore, it would follow that the overall pulmonary endothelial avidity of DT liposomes should be lower than that of ET liposomes. The fact that RT liposomes coated with anti-GPA and IgG (FIG. 11) have no pulmonary uptake supports this theory. One explanation for this seemingly self-contradictory phenomenon may be as follows. First, the fraction of anti-PECAM replaced by anti-GPA on the surface of DT liposomes is small and unlikely to significantly affect overall endothelial avidity. Second, this small proportion of anti-GPA conjugated to DT liposomes enables avid loading onto carrier RBC, unlike ET liposomes that absorb nonspecifically and passively onto RBC. This avidity-driven loading and hitchhiking on RBC reduces clearance of DT liposomes by the reticuloendothelial system, which otherwise competes with the pulmonary targeting. Third, carrier RBC squeezing through the pulmonary microvasculature bring surface-bound DT liposomes into close proximity with PECAM on the surface of EC, both boosting the efficacy of targeting and, due to the relative scarcity of anti-GPA antibody compared to anti-PECAM antibody, permitting transfer off carrier RBC and onto EC. Therefore, according to this explanation, the boosted delivery to the pulmonary vasculature by DTRH is a product of both specific EC targeting and active, transient RBC binding.

Having demonstrated high efficacy in lung targeting and transfer ratio without deleterious effect on RBC in vivo, the same DT liposome formulation was used to study kinetics over time. RBC-loaded DT liposomes were again IV injected into mice; ¹²⁵I-DT liposomes but not ⁵¹Cr-RBC rapidly accumulate in the lungs with an immediate peak at 2 min post-injection (FIG. 3C). In contrast, the blood level of ⁵¹Cr-RBC but not of ¹²⁵I-DT liposomes increases over time after injection. This indicates that DTRH does not damage carrier RBC, which return to the circulation after dissociation from DT liposomes.

The original approach for delivering nanocarriers to the vasculature using RBC hitchhiking (RH) did not involve specific ligand-mediated binding to RBC or to EC. Instead, accumulation in the target was predominantly mediated by the first pass phenomenon. In contrast, DTRH is based on specific binding of liposomes mediated by conjugated ligands. For example, FIG. 11 shows that replacing anti-PECAM with IgG in DT liposomes abrogated pulmonary delivery of liposomes by RBC. None of the untargeted liposomes demonstrated specific lung targeting, showing there was not a passive RH targeting effect with this liposome formulation. This is in contrast to the liposome formulation used in prior studies¹⁸, for which untargeted passive RH was effective. The major difference in the two liposome formulations is that the prior formulations used SATA-maleimide conjugation chemistry to link IgG molecules to the liposome, while the current study uses the more translatable copper-free click chemistry (DBCO-azide). Regardless of anti-GPA:IgG surface density, ⁵¹Cr-RBC could be found in blood and spleen, while liposomes localized to the liver and spleen. The noticeable increase in clearance from the blood, partnered with RES uptake, is proportional to the number of total antibodies (anti-GPA plus IgG) on the liposomes. Conceivably, Fc-fragments of IgG molecules exposed on the DT liposomes may activate complement and Fc-receptor bearing host defense cells thereby increasing clearance mechanisms.

In case of true DTRH as demonstrated here (where the liposome surface is composed of anti-GPA plus anti-EC), the avidity of DT liposomes to RBC, in addition to total Fc burden, may contribute to a reduction of DT liposome biocompatibility. For example, multivalent binding of DT liposomes to GPA on the RBC surface may rigidify the carrier RBC. We determined conditions for the number of surface antibodies, GPA targeting antibodies, and ratio of GPA:EC targeting antibodies for DT liposome formulations and RBC loading that do not cause RBC pathology (FIG. 2A-FIG. 2F, FIG. 3A, FIG. 8A-FIG. 8B). These parameters were further refined in vivo by analyzing RBC clearance data when 40 versus 450 RT liposomes are loaded per RBC. RBC loaded with 450 RT liposomes were eliminated instantly after injection, while loading at an order of magnitude lower was more benign in terms of effect on RBC biocompatibility (FIG. 11). When carefully considering all of these parameters, multiple in vivo studies throughout this work indicate that precisely formulated DT liposomes for DTRH avoid overt and intolerable effects on carrier RBC.

This concept is again demonstrated in FIG. 3D. Having achieved high lung targeting and transfer ratio (610) with DTRH using DT liposomes only 2.5% targeted against RBC, we hypothesized that increasing the affinity toward RBC would hitchhiking and thereby increase lung targeting. RBC loaded with DT liposomes (10%:90% for a total of 20 RBC targeting molecules and 180 EC targeting molecules) provided unparalleled ¹²⁵I-DT liposome pulmonary uptake, approaching 100% of injected dose. However, a concomitant and undesirable elevation of ⁵¹Cr-RBC was observed in the lungs (FIG. 3D, inset). Several mechanisms may explain this finding: cross-linking and rigidification of the RBC membrane by multivalent DT liposomes may provoke non-specific adhesion of RBC carriers to endothelium; or the increased avidity of DT liposomes to RBC may prevent dissociation after DT liposomes bind their pulmonary endothelial target cells. While specifically targeting RBC to the vascular cells may serve a purpose for drug delivery, in this case the elevated level of ⁵¹Cr-RBC in the lungs, and resulting low transfer ratio (120), reveals that the extraordinary pulmonary accumulation of DT liposomes is at least partially due to pathologic retention of RBC. This validates the in vitro findings demonstrating that DT liposomes coated with antibodies in a 10:90% ratio of GPA:PECAM behave more similarly to RT liposomes than ET liposomes.

### Humanizing DT Liposomes and Perfusion in Human Lungs

As presented, DT liposomes were developed in a murine model and shown to successfully deliver to the lungs with high efficacy and efficient transfer from carrier RBC. To determine the feasible utility in human therapeutics, DT liposomes were modified to target human RBC and EC, and were tested for proof-of-concept ex vivo in human lungs. DT liposomes were coated with antibodies against either GPA or RhCE surface antigens on human RBC. GPA was chosen to mimic the murine model testing, knowing that binding to GPA may induce RBC membrane rigidification.³⁵ Therefore RhCE, a ubiquitous membrane protein expressed on human RBC that does not induce rigidification, was also evaluated. An anti-human-PECAM antibody was used to target ECs, as human pulmonary EC express PECAM similarly to murine pulmonary endothelium. We used the human PECAM clone Ab62, and one of two RBC-binding antibodies, either the GPA clone CD235a, or the RhCE clone Bric69. Each liposome was coated with 100 antibodies for all experiments targeting human cells. The percent of RBC antibodies was 0, 5, 10, 25, or 100%, with the remainder directed against EC for binding studies (FIG. 4A).

These humanized DT liposomes were tested for binding and agglutination of human RBCs to anticipate their behavior in vivo, using the same techniques shown in FIG. 2A-FIG. 2F. When using an RBC-binding antibody that targets GPA, the addition of just 5% RBC-binding antibody resulted in increased loading onto RBC compared to nonspecific loading (FIG. 4A, left panel). Not surprisingly, deleterious effects on RBC were observed when 200 or more liposomes were bound per RBC when bound liposomes had 25% or more of their surface targeted to GPA. In contrast, DT liposomes against RhCE rather than GPA have little to no adverse effects on RBC, showing no significant agglutination. They also don't show increased avidity to RBC compared to nonspecific binding until approximately 25% of a liposome's surface is directed against RhCE (FIG. 4A, right panel). While liposomes directed 100% against RhCE do show significantly improved loading onto RBCs, the absolute fraction loaded is less than liposomes directed 100% against GPA. To improve on RBC loading without inducing RBC membrane pathology, an increase in the total number of antibodies on the surface of DT liposomes could be considered, mimicking the in vivo work in the murine model.

Using humanized DT liposomes that can safely and effectively load onto human RBC, we next endeavored to show that DTRH can deliver to the human pulmonary vasculature. We have previously published on testing nanoparticle binding in an ex vivo human lung model.¹⁸ Briefly, we used whole lobes from fresh human lungs that were rejected for transplantation. The lungs are perfused, ventilated, and endovascularly cannulated for infusion of nanocarriers (FIG. 4B and FIG. 4C). Human RBC from a volunteer donor of blood type matched to the test lung were prepared and labeled with ⁵¹Cr as described above, then loaded with DT liposomes. Guided by in vitro results, ¹²⁵I labeled DT liposomes with 100 surface antibodies; 5% against GPA and 95% against PECAM were perfused through the pulmonary artery (FIG. 4C). Of the initial injected dose, 27.5% of DT-liposomes remained in the lungs, while the majority of carrier RBC (84.6%) left the pulmonary circulation. A higher percentage of RBC left the pulmonary circulation compared to DT liposomes, indicating transfer of DT liposome off RBCs and retention in the pulmonary vasculature. The fact that this transfer happened on a single-pass circulation further demonstrates the transient binding of DT liposome to RBCs, compared to the more permanent binding to endothelium. Further, it provides support that DTRH will be effective in the human circulation.

### DTRH is Generalizable to Diverse Endothelial Epitopes

The preceding lung uptake studies, in both murine and human lungs, were completed with DT liposomes targeting the endothelial adhesion molecule PECAM. One goal of DTRH is that it be a generalizable method for effective, specific drug delivery. We therefore tested DTRH with the EC adhesion molecule target, ICAM. ICAM is an advantageous endothelial target since the adhesion molecule's expression, robustly expressed normally, is further enhanced by inflammation. ^{8,42,44-47} To construct DT liposomes, we were guided by the in vitro RBC binding experiments of FIG. 2A and FIG. 2B. DT liposomes were formulated with 200 total monoclonal antibodies in two specific ratios: 10%:90% (for a total of 20 Ter119 clones targeting GPA and 180 YN1 clones targeting ICAM) and 25%:95% (for a total of 50 Ter119 targeting GPA and 150 YN1 targeting ICAM). Due to the binding characteristics shown in FIG. 2C, DT liposomes formulated in both ratios (10:90 and 25:75) were chosen as likely to both load onto RBC and avoid adverse consequences to the RBC membrane.

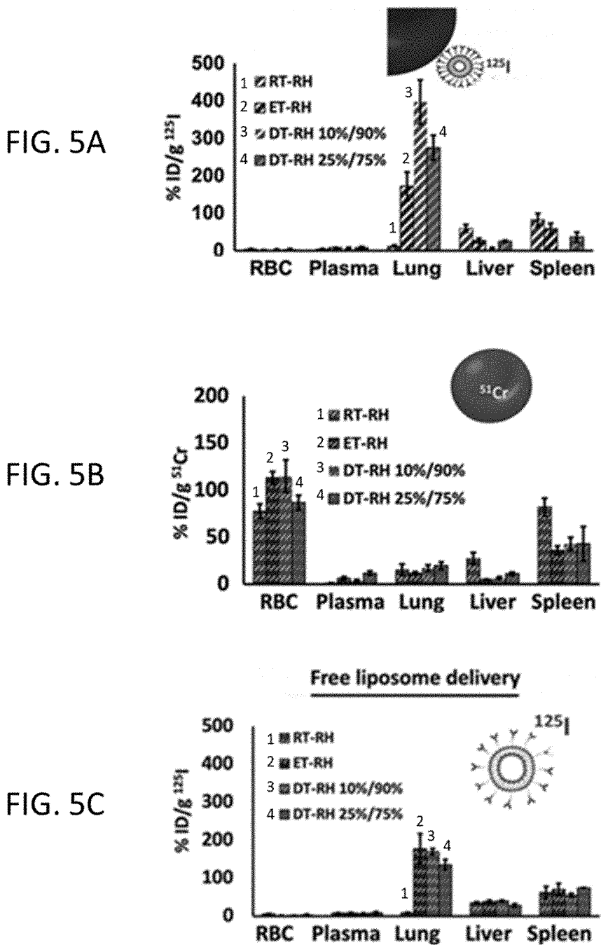

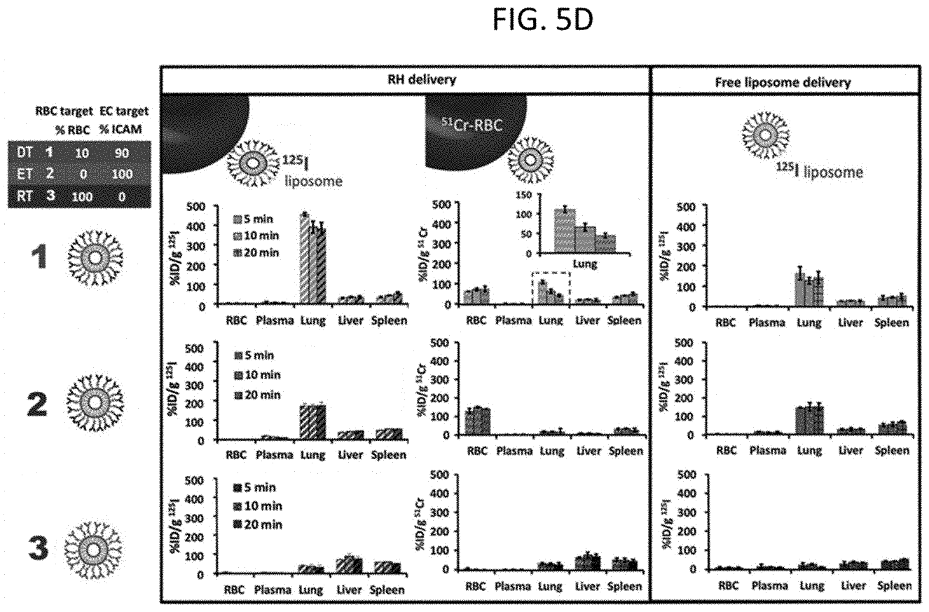

To test these DT liposomes and compare them to their cognate ET and RT versions, we first IV injected them as free liposomes, without RBC hitchhiking, and harvested organs 30 minutes later to create biodistribution plots (FIG. 5C). As anticipated, the lung accumulation of the formulations of freely delivered liposomes vary little between ET liposomes (200 anti-EC targeting antibodies) which delivered 178% (±39%) ID/g vs 10:90 DT liposomes (180 anti-EC targeting antibodies) which delivered 170% (±8.3%) ID/g. While trending lower, 25:75 DT liposomes (150 anti-EC targeting antibodies) achieved 135% ID/g (±8.3%), not statistically different (p>0.05 by student t-test) but at 135% ID/g (±8.3%) trended lower. Very little accumulation of the RT liposomes was seen in either the RBC or the lung compartments, with most of the activity located in the liver and spleen. The slightly reduced total recovery of activity (sum of total % ID=˜65%, usually >80% % ID) suggests rapid RES clearance and/or injection site retention. Thus, in the absence of RBC binding ex vivo, DT liposomes perform closely to ET liposomes in vivo, with high lung uptake. We previously hypothesized that multiple mechanisms contribute to increased uptake by DTRH in the pulmonary vasculature, in particular the close contact between DT liposomes and EC targets due to RBC squeezing through the capillary meshwork. The lack of enhanced lung delivery demonstrated with free DT liposome injection further supports the hypothesis that the improved delivery is due to RBC hitchhiking rather than in innate property of these DT liposomes.

As RBC loading is now shown to be required for the DTRH delivery effect, both formulations were first bound to RBC ex vivo before IV injection and 30 min biodistribution. Both ⁵¹Cr-RBC and ¹²⁵I-DT liposomes were traced. Neither DTRH formulation caused RBC retention in the lungs (FIG. 5A), thus illustrating the relative safety of DTRH. Most importantly, FIG. 5B shows that the optimal DTRH formulation (10%:90%) improves lung delivery by >2.5-fold compared to the predicate technology of ET-RH (used to approximate passive, nonspecific RBC loading). Additionally, comparing ET-RH liposome delivery to the lungs (FIG. 5B) vs free ET liposome delivery (FIG. 5C), we see once again that passive RH does not benefit lung targeting for this liposome formulation. Further, the reduction of lung targeting in 25%:75% DTRH vs 10%:90% DTRH (FIG. 5C) emphasizes the need for optimization of antibody ratios based on specific targeting molecule of interest.

Having observed that the 10%:90% DTRH formulation yielded ˜400% ID/g in the lung, higher than any ICAM-targeted formulation previously studied, we next investigated the kinetics of this formulation. We aimed to determine not only DT liposome association with tissue, but also the kinetics of dissociation between the liposomes and RBCs. In the table in FIG. 5D, % RBC and % ICAM refer to the % of 200 total antibodies/liposome surface. Therefore, formulation 1 (row 1) refers to free DT liposomes and DTRH liposomes; formulation 2 refers to free ET liposomes and ET-RH liposomes; and formulation 3 refers to free RT liposomes and RT-RH liposomes. Note that the first column in FIG. 5D traces the ⁵¹Cr signal of carrier RBC and the second column traces ¹²⁵I-liposomes from the same animals. The separated far right column traces free ¹²⁵I-liposomes from separate experimental animals. Mice were IV injected with the specified liposome formulation and sacrificed at 5, 10 and 20 mins, shown left to right in each organ grouping.

Analyzing the data of FIG. 5D, several key features are notable. Kinetics of DTRH are shown in row 1. From this, it is evident that DT liposomes reached their peak concentration in the lungs at <5 min (confirming results with an alternative CAM target in FIG. 3A-FIG. 3D), remained statistically unchanged over time, and this value is >2.5 fold compared higher than achieved with ET-RH. Importantly, while this high lung retention remained stable, the ⁵¹Cr signal (inset) indicated that carrier RBC separated from their cargo and left the lung over time. This further validates that DT liposomes transiently bind carrier RBC and, when avidity to RBC is low enough, dissociate from RBC after reaching the pulmonary endothelium. Neither ET-RH now RT-RH displayed this change in organ deposition of RBC over time. Lung accumulation of liposomes by ET-RH, compared to free ET liposomes (row 2) indicated that in this case, passive RH slightly increased lung uptake (<1.5×), the only instance in which we could detect a passive RH effect, further demonstrating the inconsistency of passive RH. Finally, RT liposomes (row 3), that did not target the lungs were quickly cleared from the circulation. Thus, these data show that DTRH works with multiple endothelial epitopes, achieves rapid delivery to the lungs that is up to 2.5× higher than simple endothelial targeting, and allows RBC to dissociate from their DT liposome cargo to leave the lungs.

### DTRH Enhances Cell-Type-Targeting

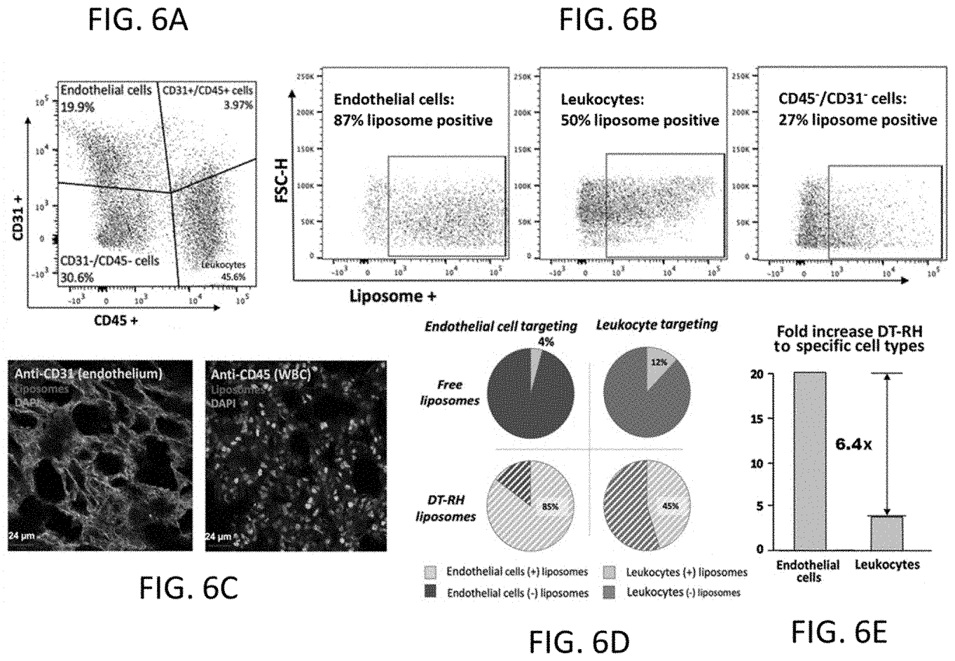

The experiments above show that DTRH can increase organ-targeting, but give no insight into precisely where or to what cell subtypes the liposomes localize. DT liposomes were reformulated to include a fluorescent lipid for histology and flow cytometry analysis. We compared these DTRH liposomes versus identical “free” DT liposomes (not loaded onto RBC) using the same dual targeting ratio of 10%:90% (RBC-binding vs ICAM-binding antibody) used in FIG. 5A-FIG. 5D. Mice were injected with these DTRH or DT liposomes and 30 minutes later the lungs were harvested, with some going towards histology, and the remainder dissociated into a single cell suspension, and analyzed by flow cytometry. Endothelial cells were defined as CD31+/CD45- and leukocytes were defined as CD31-/CD45+(cell classification scheme shown in FIG. 6A).

Lung histology (FIG. 6C) indicated that the liposomes successfully reach the alveolar capillary and interstitial space, and appear to be localized both with endothelial cells and leukocytes. To quantify this, we analyzed the fraction of liposome-positive cells found in flow cytometry. Of the endothelial cell population, 85% of cells were bound by liposomes delivered by DTRH, a 20-fold increase from the 4% of endothelial cells bound by free DT liposomes which were injected without being adsorbed onto RBCs (FIG. 6D left panel, vs bottom). This 20-fold increase far exceeds the 2.5-fold increase in overall lung targeting achieved by DTRH compared to free EC liposome delivery (FIG. 1C), demonstrating that DTRH liposomes have an extra tropism for endothelial cells compared to other resident lung cells. Similarly, the leukocyte population was 45% positive for liposomes delivered by DTRH compared to 12% by DT liposomes (FIG. 6D right panel top vs bottom), a 2.75-fold advantage that is largely explained by the overall increase in liposome delivery. If we divide DTRH's advantages in endothelial targeting, we find that the endothelial-over-leukocyte preference is 6.4-fold. Therefore, DTRH not only increased the organ-targeting by >2.5-fold, but within the target organ (lung), DTRH improved cell-type targeting to endothelial cells by >6-fold, a cumulative increase in pulmonary endothelial targeting of 16-fold.

The goal of nanomedicine has long been to localize drug-loaded nanocarriers to a specific organ and/or cell type. The field has made tremendous progress towards this goal, in large part from conjugating ligands onto the surface of nanocarriers. However, in nearly every case, far less than half the nanocarrier ends up in the target organ (unless the target organ is the liver), with values of <1% being common for many target tissues such as brain and tumor.

DTRH provides synergy between dual ligand targeting and cell-mediated delivery and has advantages over predicate technologies:

First, compared to passive RH, DTRH dramatically increased the efficiency of adsorbing nanocarriers onto the RBC surface. As shown in FIG. 2E-FIG. 2F, compared to passive RH, DTRH had 43-fold more nanocarrier signal on RBCs. Importantly, in prior work on passive RH¹⁸, most nanocarrier types had <10% of the nanocarriers adsorb onto the RBC, which means 90% of the nanocarrier is lost in preparation, thus increasing material costs 10-fold. DTRH's improved efficiency of nanocarrier loading onto RBCs thus makes the technology much less costly.

Second, DTRH markedly improved organ-targeting. FIG. 1C compares DTRH to other carriers and free drug, with the metric being the percent of the injected dose in the target organ (lungs) after 30 minutes in mice. Sequential dual targeting/DTRH delivered to the target organ (lungs)>65% of the injected dose of nanocarriers. Notably, this was 2.5× better than achieved with single-antibody-targeting (labeled as ET L) using the most studied group of lung-targeting antibodies, anti-CAMs. It was also >2× better than passive RH (no RBC-binding antibody), even when the RH included an anti-CAM antibody. Importantly, DTRH accumulated at >650-fold higher than a hydrophilic small molecule drug (DTPA). Lastly, DTRH is the only intravascularly-delivered nanotechnology that has been shown to deliver liposomes to the lungs such that the majority (>50%) of the liposomes end up in the target organ.

Third, DTRH also dramatically improved cell-type-targeting within the target organ. FIG. 6E shows that within the target organ (lungs), DTRH nanocarriers had a 6.4-fold higher preference for the target cell type (endothelial cells) than did single-targeted nanocarriers (with anti-CAM antibodies, but not adsorbed to RBCs). This was determined by flow cytometry, comparing the fraction of nanocarrier-positive endothelial cells vs nanocarrier-positive non-endothelial cells, which were almost exclusively leukocytes (CD45+). It may at first seem surprising that a significant fraction of anti-CAM liposomes are taken up by pulmonary leukocytes, as anti-CAM liposomes have been assumed for decades to exclusively target endothelial cells within the lungs.^{6,8} However, it is well documented that alveolar capillaries have abundant “marginated” neutrophils and monocytes that reside in the alveolar capillary lumen^{48,49}, and these cells express CAMs.⁵⁰ Depending on the cargo drug and target disease, it may be highly advantageous for lung-targeted nanocarriers to have much greater specificity for endothelial cells (i.e., maximize the fraction of nanocarrier-positive cells that are endothelial). DTRH provides such increased cell-type specificity, improving it by >6-fold.

Fourth, DTRH can increase the types of nanocarriers that work with RBC-hitchhiking. In previous work¹⁸, while passive RH improved lung uptake at least to some extent on seven tested types of nanocarriers, only two of those produced lung localization comparable to anti-CAM nanocarriers, with the others displaying at least 5-fold lower uptake. The mechanism underlying passive RH's variability between nanocarriers is still unknown. By using a more defined binding system for RH, namely the two-antibody system of DTRH, we could convert a nanocarrier that does not work for passive RH into one that does benefit from RH's several advantages. Indeed, the nanocarriers employed in this Example do not work with passive RH, but do work with DTRH. The nanocarriers employed were as close to clinical application as possible. For the carrier itself, we chose liposomes, since they are the most clinically employed nanocarrier. These liposomes were conjugated to IgG molecules (the most common ligand employed clinically) via copper-free “click chemistry”, chosen because of its advantages for scale-up manufacturing (near 100% efficiency, stoichiometric addition, with no toxins to purify after). When adsorbed passively onto RBCs, these liposomes did not show a significant RH-effect or lung uptake (FIG. 2B). However, these liposomes, in DTRH format, had 65% of the injected dose go to the lungs. In our prior work¹⁸, we found that liposomes conjugated via SATA-maleimide chemistry do work with passive RH, though delivering to the lungs at half the rate as DTRH. The fact that passive RH does not work for one out of two common conjugation chemistries illustrates how difficult it will be to further develop passive RH, with its unknown mechanism of RBC-nanocarrier binding. By contrast, DTRH works via a well-defined mechanism of binding, and can broaden the range of applicable nanocarriers.

In addition to the above advantages, another potential benefit of DTRH is that it can be rationally engineered, rather than relying on unknown mechanisms like passive RH. DTRH is composed of multiple components with easily quantifiable properties, namely one ligand that binds the mobile cell and another that binds the target cell. There is tremendous design flexibility, as the ligands can be changed in terms of: target epitopes (e.g., here we showed DTRH works with both anti-ICAM and -PECAM antibodies), absolute number, ratio of the two ligands, specific affinity (e.g., changing to a different antibody clone), and type of ligand (e.g., changing from monoclonal antibody to the single chain variable (scFv) format).

In summary, DTRH combines ligand-targeted and cell-mediated nanocarrier delivery that provides significant advantages over prior technologies such as passive RBC-hitchhiking and single-antibody targeting with anti-CAM antibodies. First, DTRH improves the adsorption efficiency compared to passive RH. Second, DTRH improves organ-targeting by >2-fold over passive RH and single-antibody-targeting, with delivering as much as 65% of the injected dose to the target organ. Third, DTRH improves cell-type-targeting, >6-fold over single-targeting. Fourth, DTRH can work with nanocarriers that do not work with passive RH.

### REFERENCES

1 Torchilin, V. P., Khaw, B. A., Smirnov, V. N. & Haber, E. Preservation of antimyosin antibody activity after covalent coupling to liposomes. Biochem Biophys Res Commun 89, 1114-1119, doi:10.1016/0006-291x(79)92123-5 (1979).
2 Shamay, Y. et al. P-selectin is a nanotherapeutic delivery target in the tumor microenvironment. Sci Transl Med 8, 345ra387, doi:10.1126/scitranslmed.aaf7374 (2016).
3 Keshavarz, A. et al. CAR, a Homing Peptide, Prolongs Pulmonary Preferential Vasodilation by Increasing Pulmonary Retention and Reducing Systemic Absorption of Liposomal Fasudil. Mol Pharm 16, 3414-3429, doi:10.1021/acs.molpharmaceut.9b00208 (2019).
4 Wilhelm, S. et al. Analysis of nanoparticle delivery to tumours. Nature Reviews Materials 1, 16014, doi:10.1038/natrevmats.2016.14 (2016).
5 Maruyama, K. et al. Characterization of in vivo immunoliposome targeting to pulmonary endothelium. J Pharm Sci 79, 978-984, doi:10.1002/jps.2600791107 (1990).
6 Muzykantov, V. R. et al. Streptavidin facilitates internalization and pulmonary targeting of an anti-endothelial cell antibody (platelet-endothelial cell adhesion molecule 1): a strategy for vascular immunotargeting of drugs. Proc Natl Acad Sci USA 96, 2379-2384, doi:10.1073/pnas.96.5.2379 (1999).
7 Hood, E. D. et al. Antioxidant protection by PECAM-targeted delivery of a novel NADPH-oxidase inhibitor to the endothelium in vitro and in vivo. Journal of controlled release: official journal of the Controlled Release Society 163, 161-169, doi:10.1016/j.jconre1.2012.08.031 (2012).
8 Brenner, J. S. et al. Mechanisms that determine nanocarrier targeting to healthy versus inflamed lung regions. Nanomedicine 13, 1495-1506, doi:10.1016/j.nano.2016.12.019 (2017).
9 Zhang, C. et al. Direct Macromolecular Drug Delivery to Cerebral Ischemia Area using Neutrophil-Mediated Nanoparticles. Theranostics 7, 3260-3275, doi:10.7150/thno.19979 (2017).
10 Klyachko, N. L. et al. Macrophages with cellular backpacks for targeted drug delivery to the brain. Biomaterials 140, 79-87, doi:10.1016/j.biomaterials.2017.06.017 (2017).
11 Anselmo, A. C. et al. Monocyte-mediated delivery of polymeric backpacks to inflamed tissues: a generalized strategy to deliver drugs to treat inflammation. Journal of controlled release: official journal of the Controlled Release Society 199, 29-36, doi:10.1016/j.jconre1.2014.11.027 (2015).
12 Fraternale, A., Rossi, L. & Magnani, M. Encapsulation, metabolism and release of 2-fluoro-ara-AMP from human erythrocytes. Biochim Biophys Acta 1291, 149-154, doi:10.1016/0304-4165(96)00059-1 (1996).
13 Rossi, L. et al. Heterodimer-loaded erythrocytes as bioreactors for slow delivery of the antiviral drug azidothymidine and the antimycobacterial drug ethambutol. AIDS Res Hum Retroviruses 15, 345-353, doi:10.1089/088922299311312 (1999).
14 Magnani, M., Pierige, F. & Rossi, L. Erythrocytes as a novel delivery vehicle for biologics: from enzymes to nucleic acid-based therapeutics. Ther Deliv 3, 405-414, doi:10.4155/tde.12.6 (2012).
15 Sharma, R. et al. Mechanisms of enhanced neutralization of botulinum neurotoxin by monoclonal antibodies conjugated to antibodies specific for the erythrocyte complement receptor. Mol Immunol 57, 247-254, doi:10.1016/j.molimm.2013.09.005 (2014).
16 Zaitsev, S. et al. Human complement receptor type 1-directed loading of tissue plasminogen activator on circulating erythrocytes for prophylactic fibrinolysis. Blood 108, 1895-1902, doi:10.1182/blood-2005-11-012336 (2006).
17 Lindorfer, M. A. et al. Targeting of Pseudomonas aeruginosa in the bloodstream with bispecific monoclonal antibodies. J Immunol 167, 2240-2249, doi:10.4049/jimmunol.167.4.2240 (2001).
18 Brenner, J. S. et al. Red blood cell-hitchhiking boosts delivery of nanocarriers to chosen organs by orders of magnitude. Nat Commun 9, 2684, doi:10.1038/s41467-018-05079-7 (2018).
19 Anselmo, A. C. et al. Delivering nanoparticles to lungs while avoiding liver and spleen through adsorption on red blood cells. ACS nano 7, 11129-11137, doi:10.1021/nn404853z (2013).
20 Pan, D. et al. The Effect of Polymeric Nanoparticles on Biocompatibility of Carrier Red Blood Cells. PLoS One 11, e0152074, doi:10.1371/journal.pone.0152074 (2016).
21 Glassman, P. M. et al. Vascular Drug Delivery Using Carrier Red Blood Cells: Focus on RBC Surface Loading and Pharmacokinetics. Pharmaceutics 12, doi:10.3390/pharmaceutics12050440 (2020).
22 Villa, C. H., Anselmo, A. C., Mitragotri, S. & Muzykantov, V. Red blood cells: Supercarriers for drugs, biologicals, and nanoparticles and inspiration for advanced delivery systems. Adv Drug Deliv Rev 106, 88-103, doi:10.1016/j.addr.2016.02.007 (2016).
23 Fromen, C. A. et al. Evaluation of Receptor-Ligand Mechanisms of Dual-Targeted Particles to an Inflamed Endothelium. Bioeng Transl Med 1, 103-115, doi:10.1002/btm2.10008 (2016).
24 Eniola, A. O., Willcox, P. J. & Hammer, D. A. Interplay between rolling and firm adhesion elucidated with a cell-free system engineered with two distinct receptor-ligand pairs. Biophys J 85, 2720-2731, doi:10.1016/s0006-3495(03)74695-5 (2003).
25 Weller, G. E., Villanueva, F. S., Tom, E. M. & Wagner, W. R. Targeted ultrasound contrast agents: in vitro assessment of endothelial dysfunction and multi-targeting to ICAM-1 and sialyl Lewisx. Biotechnol Bioeng 92, 780-788, doi:10.1002/bit.20625 (2005).
26 Sun, D. et al. Superior sensitivity of novel molecular imaging probe: simultaneously targeting two types of endothelial injury markers. FASEB J 24, 1532-1540, doi:10.1096/fj.09-148981 (2010).
27 Omolola Eniola, A. & Hammer, D. A. In vitro characterization of leukocyte mimetic for targeting therapeutics to the endothelium using two receptors. Biomaterials 26, 7136-7144, doi:10.1016/j.biomaterials.2005.05.005 (2005).
28 McAteer, M. A. et al. Magnetic resonance imaging of endothelial adhesion molecules in mouse atherosclerosis using dual-targeted microparticles of iron oxide. Arterioscler Thromb Vasc Biol 28, 77-83, doi:10.1161/ATVBAHA.107.145466 (2008).
29 Rafat, M., Rotenstein, L. S., Hu, J. L. & Auguste, D. T. Engineered endothelial cell adhesion via VCAM1 and E-selectin antibody-presenting alginate hydrogels. Acta Biomater 8, 2697-2703, doi:10.1016/j.actbio.2012.04.010 (2012).
30 Guo, P. et al. Dual complementary liposomes inhibit triple-negative breast tumor progression and metastasis. Sci Adv 5, eaav5010, doi:10.1126/sciadv.aav5010 (2019).
31 Papademetriou, I. T., Garnacho, C., Schuchman, E. H. & Muro, S. In vivo performance of polymer nanocarriers dually-targeted to epitopes of the same or different receptors. Biomaterials 34, 3459-3466, doi:10.1016/j.biomaterials.2013.01.069 (2013).
32 Ansar, M., Serrano, D., Papademetriou, I., Bhowmick, T. K. & Muro, S. Biological functionalization of drug delivery carriers to bypass size restrictions of receptor-mediated endocytosis independently from receptor targeting. ACS nano 7, 10597-10611, doi:10.1021/nn404719c (2013).
33 Li, X. et al. The enhanced treatment efficacy of invasive brain glioma by dual-targeted artemether plus paclitaxel micelles. Artif Cells Nanomed Biotechnol 48, 983-996, doi:10.1080/21691401.2020.1773489 (2020).
34 Krasavin, M. et al. Combining carbonic anhydrase and thioredoxin reductase inhibitory motifs within a single molecule dramatically increases its cytotoxicity. J Enzyme Inhib Med Chem 35, 665-671, doi:10.1080/14756366.2020.1734800 (2020).
35 Villa, C. H. et al. Biocompatible coupling of therapeutic fusion proteins to human erythrocytes. Blood Adv 2, 165-176, doi:10.1182/bloodadvances.2017011734 (2018).
36 Glassman, P. M. et al. Targeting drug delivery in the vascular system: Focus on endothelium. Adv Drug Deliv Rev 157, 96-117, doi:10.1016/j.addr.2020.06.013 (2020).
37 Hood, E. D. et al. Vascular targeting of radiolabeled liposomes with bio-orthogonally conjugated ligands: single chain fragments provide higher specificity than antibodies. Bioconjug Chem, doi:10.1021/acs.bioconjchem.8b00564 (2018).
38 Hood, E. D. et al. Endothelial targeting of nanocarriers loaded with antioxidant enzymes for protection against vascular oxidative stress and inflammation. Biomaterials 35, 3708-3715, doi:10.1016/j.biomaterials.2014.01.023 (2014).
39 Howard, M. D., Greineder, C. F., Hood, E. D. & Muzykantov, V. R. Endothelial targeting of liposomes encapsulating SOD/catalase mimetic EUK-134 alleviates acute pulmonary inflammation. Journal of controlled release: official journal of the Controlled Release Society 177, 34-41, doi:10.1016/j.jconre1.2013.12.035 (2014).
40 Khoshnejad, M. et al. Vascular Accessibility of Endothelial Targeted Ferritin Nanoparticles. Bioconjug Chem 27, 628-637, doi:10.1021/acs.bioconjchem.5b00641 (2016).
41 Kiseleva, R. et al. Mechanism of Collaborative Enhancement of Binding of Paired Antibodies to Distinct Epitopes of Platelet Endothelial Cell Adhesion Molecule-1. PLoS One 12, e0169537, doi:10.1371/journal.pone.0169537 (2017).
42 Shuvaev, V. V. et al. Size and targeting to PECAM vs ICAM control endothelial delivery, internalization and protective effect of multimolecular SOD conjugates. Journal of controlled release: official journal of the Controlled Release Society 234, 115-123, doi:10.1016/j.jconre1.2016.05.040 (2016).
43 Howard, M. et al. Vascular targeting of nanocarriers: perplexing aspects of the seemingly straightforward paradigm. ACS nano 8, 4100-4132, doi:10.1021/nn500136z (2014).
44 Marcos-Contreras, O. A. et al. Selective targeting of nanomedicine to inflamed cerebral vasculature to enhance the blood-brain barrier. Proc Natl Acad Sci USA 117, 3405-3414, doi:10.1073/pnas.1912012117 (2020).
45 Marcos-Contreras, O. A. et al. Combining vascular targeting and the local first pass provides 100-fold higher uptake of ICAM-1-targeted vs untargeted nanocarriers in the inflamed brain. Journal of controlled release: official journal of the Controlled Release Society 301, 54-61, doi:10.1016/j.jconre1.2019.03.008 (2019).
46 Ferrer, M. C. et al. Icam-1 targeted nanogels loaded with dexamethasone alleviate pulmonary inflammation. PLoS One 9, e102329, doi:10.1371/journal.pone.0102329 (2014).
47 Chacko, A. M., Hood, E. D., Zern, B. J. & Muzykantov, V. R. Targeted Nanocarriers for Imaging and Therapy of Vascular Inflammation. Current opinion in colloid & interface science 16, 215-227, doi:10.1016/j.cocis.2011.01.008 (2011).
48 Yipp, B. G. et al. The Lung is a Host Defense Niche for Immediate Neutrophil-Mediated Vascular Protection. Sci Immunol 2, doi:10.1126/sciimmunol.aam8929 (2017).
49 Granton, E., Kim, J. H., Podstawka, J. & Yipp, B. G. The Lung Microvasculature Is a Functional Immune Niche. Trends Immunol 39, 890-899, doi:10.1016/j.it.2018.09.002 (2018).
50 Woodfin, A. et al. ICAM-1-expressing neutrophils exhibit enhanced effector functions in murine models of endotoxemia. Blood 127, 898-907, doi:10.1182/blood-2015-08-664995 (2016).
51 Allen, T. M. & Cullis, P. R. Liposomal drug delivery systems: from concept to clinical applications. Adv Drug Deliv Rev 65, 36-48, doi:10.1016/j.addr.2012.09.037 (2013).
52 Greineder, C. F. et al. Site-Specific Modification of Single-Chain Antibody Fragments for Bioconjugation and Vascular Immunotargeting. Bioconjug Chem 29, 56-66, doi:10.1021/acs.bioconjchem.7b00592 (2018).
53 Bain, B. J., Bates, I., Laffan, M. A. & Lewis, S. M. Dacie and Lewis practical haematology. (2017).
54 Vallabhajosyula, P. et al. Ex Vivo Lung Perfusion Model to Study Pulmonary Tissue Extracellular Microvesicle Profiles. Ann Thorac Surg 103, 1758-1766, doi:10.1016/j.athoracsur.2016.11.074 (2017).

Each and every patent, patent application, and publication, including websites and other publications cited throughout the specification, is incorporated herein by reference. While the invention has been described with reference to particular embodiments, it will be appreciated that modifications can be made without departing from the spirit of the invention. Such modifications are intended to fall within the scope of the appended claims.

1. A composition comprising dual-targeted nanoparticles having a first targeting moiety and a second targeting moiety, wherein said first targeting moiety is a red blood cell (RBC)-targeting moiety.

2. The composition according to claim 1, further comprising RBCs bound to the nanoparticles via said first targeting moiety.

3. The composition according to claim 1, wherein said first and/or second targeting moiety is an antibody or antibody fragment, carbohydrate, carbohydrate-binding compound, peptide, nucleic acid, or aptamer.

4. The composition according to claim 1, wherein the nanoparticles are liposomes, nanogels, or polymeric nanoparticles.

5. The composition according to claim 1, wherein the first targeting moiety is an antibody specific for a glycophorin, optionally glycophorin A or Band 3, or an Rh antigen, optionally RhCE.

6. The composition according to claim 1, wherein the second targeting moiety is specific for vascular endothelial cells, intravascular leukocytes, cells of reticuloendothelial system, an immune cell, cells and tissues accessible to RBC under pathological conditions such as hemorrhage and thrombosis, an infectious microorganism, or cancer.

7. The composition according to claim 1, wherein the second targeting moiety is specific for ICAM-1, PECAM-1, VCAM-1, transferrin receptor, or ACE.

8. The composition according to claim 1, wherein the nanoparticles have a polyethylene glycol (PEG) coating.

9. The composition according to claim 1, wherein the compositions are characterized by having at least one of
(a) about 50 to about 200 bound nanoparticles per RBC;
(b) about 5 to about 350 of said first targeting moiety per nanoparticle;
(c) about 2.5% to about 25% of targeting moieties on the nanoparticle surface comprise said first targeting moiety;
(d) a total number of first and second targeting moieties per nanoparticle in the range or about 5 to about 350; and
(e) a particle size diameter of about 10 nm to about 1,000 nm.

10. The composition according to claim 1, wherein the nanoparticles are loaded with a drug.

11. A pharmaceutical composition comprising an aqueous suspension comprising the composition according to claim 1.

12. A method for delivering a drug to a mammalian subject having a disease, the method comprising administering to a subject in need thereof the pharmaceutical composition according to claim 11.

13. The method according to claim 12, wherein
(a) the disease is ARDS, pulmonary arterial hypertension, pneumonia, interstitial lung disease, idiopathic pulmonary fibrosis, post-pulmonary embolism, pulmonary capilliaritis syndrome, stroke, emphysema, lung edema, or a viral or microbial infection;
(b) the disease is ARDS and wherein said drug is one of more of albuterol, dexamethasone, and palifermin; and/or
(c) the disease involves a selected mammalian organ, and said composition is administered intra-arterially or intravenously.

14. The method according to claim 12, wherein said drug is a therapeutic drug, a prophylactic drug, an imaging or diagnostic drug, an anti-rejection drug, an anti-inflammatory agent, a pro-angiogenic factor, an anti-edema agent, or an agent that prevents ischemia-reperfusion injury.

15. The method according to claim 12, wherein the drug is delivered to a target organ, optionally the lungs, brain, or heart.

16. The method according to claim 12, wherein the composition comprising the dual-targeted nanoparticles is administered to a patient and circulating RBCs in the bloodstream of the patient bind to the nanoparticles via the first targeting moiety.

17. The according to claim 12, comprising contacting the dual-targeted nanoparticles with RBCs ex vivo to bind RBCs to the nanoparticles via the first targeting moiety prior to administration to the subject, wherein the RBCs are present in a sample obtained from the patient or an autologous blood donor.

18. The method according to claim 17, further comprising separating or enriching RBCs present in the sample prior to binding to the dual-targeted nanoparticles.

19. The method according to claim 12, wherein the dual-targeted nanoparticles are administered intravenously or intraarterially.

20. A method of generating ex vivo a composition comprising dual-targeted nanoparticles having a first targeting moiety and a second targeting moiety, wherein said first targeting moiety is a red blood cell (RBC)-targeting moiety, the method comprising contacting the dual-targeted nanoparticles with RBCs, resulting in the dual-targeted nanoparticles being bound to the RBCs.
